(19) United States
(12) Patent Application Publication
(10) Pub. No.: US 2026/0004485 A1
(43) Pub. Date: Jan. 1, 2026

(54) INFORMATION PROCESSING DEVICE, INFORMATION PROCESSING METHOD, AND NON-TRANSITORY COMPUTER-READABLE MEDIUM

(71) Applicant: NIHON KOHDEN CORPORATION, Tokyo (JP)

(72) Inventor: Kou ADACHI, Saitama (JP)

(73) Assignee: NIHON KOHDEN CORPORATION, Tokyo (JP)

(21) Appl. No.: 19/242,161

(22) Filed: Jun. 18, 2025

(30) Foreign Application Priority Data
Jun. 26, 2024 (JP) 2024-102825

(51) Int. Cl. G06T 11/20; A61B 5/00; G16H 50/30

(52) U.S. Cl. CPC G06T 11/206; A61B 5/743; G16H 50/30; G06T 2210/41

(57) ABSTRACT

An information processing device includes at least one processor, and one or more memories configured to store at least one instruction to be executed by the at least one processor. When the at least one instruction is executed by the processor, the information processing device is configured to generate, based on physiological information on a subject, a plurality of pieces of change tendency information indicating a change tendency of the physiological information during one period or a change tendency of the physiological information from one period to an other period, and generate display data in which the plurality of pieces of change tendency information are displayed side by side or the plurality of pieces of change tendency information are displayed so as to be overlapped with each other.

### CROSS-REFERENCE TO RELATED APPLICATIONS

This application is based on and claims priority under 35 USC 119 from Japanese Patent Application No. 2024-102825 filed on Jun. 26, 2024, the entire content of which is incorporated herein by reference.

### TECHNICAL FIELD

The present disclosure relates to an information processing device, an information processing method, and a non-transitory computer readable medium.

### BACKGROUND ART

JP2019-150560A discloses a device that displays physiological information on a subject that changes over time and an arrow indicating an increase or decrease in a value of the physiological information.

In order to accurately recognize a condition of the subject, it is important to grasp not only the physiological information on the subject but also a change tendency of the physiological information on the subject, but it may be difficult to immediately grasp the change tendency only by glancing the physiological information on the subject.

### SUMMARY OF INVENTION

Aspect of non-limiting embodiments of the present disclosure relates to provide an information processing device, an information processing method, and a non-transitory computer readable medium capable of supporting a user such as a medical worker to intuitively and accurately grasp a change tendency of physiological information on a subject.

Aspects of certain non-limiting embodiments of the present disclosure address the features discussed above and/or other features not described above. However, aspects of the non-limiting embodiments are not required to address the above features, and aspects of the non-limiting embodiments of the present disclosure may not address features described above.

According to an aspect of the present disclosure, there is provided an information processing device including:
at least one processor; and
one or more memories configured to store at least one instruction to be executed by the at least one processor,
in which when the at least one instruction is executed by the processor, the information processing device is configured to:
   generate, based on physiological information on a subject, a plurality of pieces of change tendency information indicating a change tendency of the physiological information during one period or a change tendency of the physiological information from one period to an other period; and
   generate display data in which the plurality of pieces of change tendency information are displayed side by side or the plurality of pieces of change tendency information are displayed so as to be overlapped with each other.

According to an aspect of the present disclosure, there is provided an information processing method executed by an information processing device, the information processing method including:
generating, based on physiological information on a subject, a plurality of pieces of change tendency information indicating a change tendency of the physiological information during one period or a change tendency of the physiological information from one period to another period; and
generating display data in which the plurality of pieces of change tendency information are displayed side by side or the plurality of pieces of change tendency information are displayed so as to be overlapped with each other.

According to an aspect of the present disclosure, there is provided a non-transitory computer-readable medium storing a computer program including at least one instruction executed by one or more processors of an information processing device, the computer program causing, when the at least one instruction is executed by the one or more processors, the information processing device to:
generate, based on physiological information on a subject, a plurality of pieces of change tendency information indicating a change tendency of the physiological information during one period or a change tendency of the physiological information from one period to another period, and
generate display data in which the plurality of pieces of change tendency information are displayed side by side or the plurality of pieces of change tendency information are displayed so as to be overlapped with each other.

### BRIEF DESCRIPTION OF DRAWINGS

Exemplary embodiment(s) of the present invention will be described in detail based on the following figures, wherein:

FIG. 1 is a schematic diagram of an information processing system according to a first embodiment;

FIG. 2 is a flowchart illustrating an example of an information processing method until an image based on display data is displayed on a display unit of an information processing device;

FIG. 3 illustrates an example of the image based on the display data;

FIG. 4 is a flowchart illustrating an example of an information processing method until an image based on display data is displayed on the display unit of the information processing device;

FIG. 5 illustrates an example of the image based on the display data;

FIG. 6 illustrates an example of an image based on the display data;

FIG. 7 illustrates an example of an image based on the display data;

FIG. 8 is a schematic diagram of an information processing system according to a second embodiment;

FIG. 9 illustrates an example of an image based on the display data;

FIG. 10 illustrates an example of an image based on the display data;

FIG. 11 illustrates an example of an image based on the display data;

FIG. 12 illustrates an example of an image based on the display data;

FIG. 13 illustrates an example of an image based on the display data;

FIG. 14 illustrates an example of an image based on the display data; and

FIG. 15 illustrates an example of an image based on the display data.

### DESCRIPTION OF EMBODIMENTS

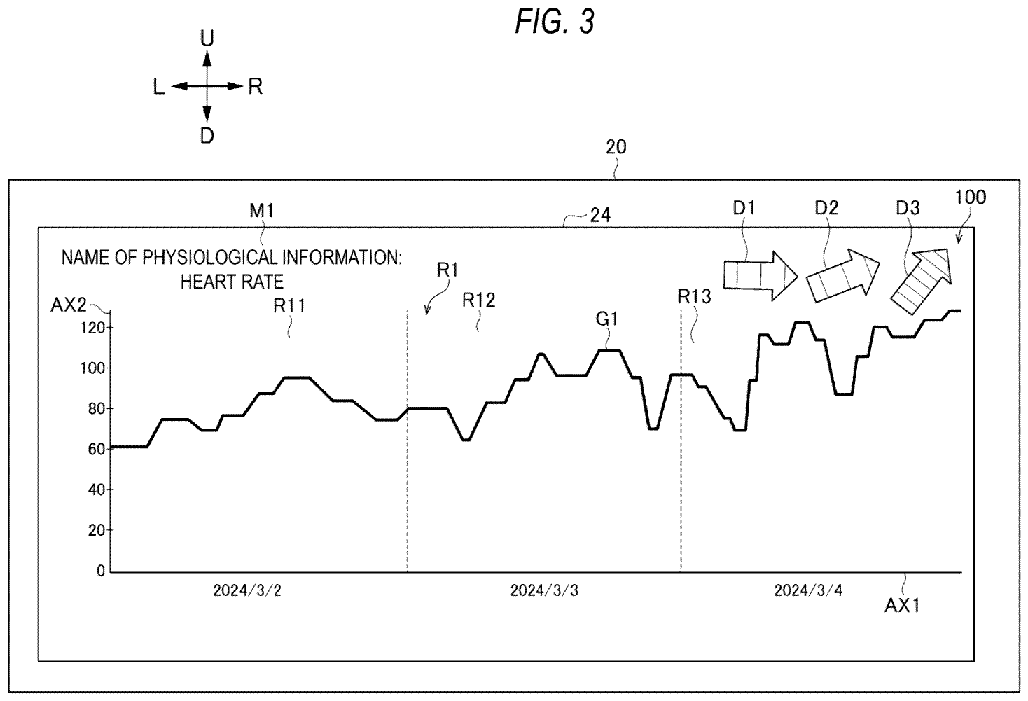

Hereinafter, examples of embodiments of the present disclosure will be described with reference to the drawings. In the description of the present embodiment, a “left-right direction” and an “up-down direction” may be appropriately referred to for convenience of description. These directions are relative directions set for a display unit 24 of an information processing device 20 illustrated in FIGS. 3, 5, 6, 7, 9, and 10 to 15. Here, the “left-right direction” is a direction including a “left direction” and a “right direction”. The “up-down direction” is a direction including an “up direction” and a “down direction”. A reference character U illustrated in each drawing indicates the up direction. A reference character D indicates the down direction. A reference character L indicates the left direction. A reference character R indicates the right direction.

### First Embodiment

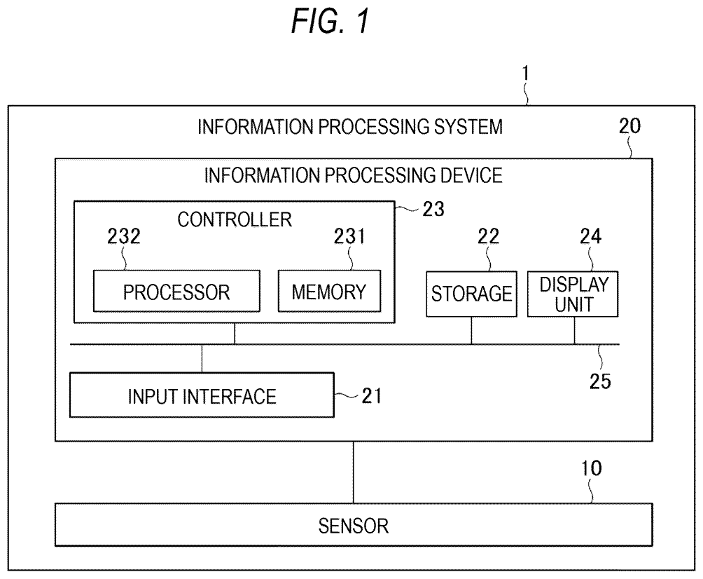

An information processing system 1 according to the present embodiment will be described with reference to FIG. 1. FIG. 1 is a schematic diagram of the information processing system 1 according to a first embodiment. The information processing system 1 is used in, for example, a medical facility such as a hospital. As illustrated in FIG. 1, the information processing system 1 can include a sensor 10 and the information processing device 20. The sensor 10 and the information processing device 20 are connected by wire or wirelessly.

The sensor 10 is configured to measure physiological information on a subject. The sensor 10 can include, for example, a blood pressure sensor, a respiration sensor, an SpO2 sensor, a body temperature sensor, a heart rate sensor, and the like. The blood pressure sensor is configured to measure a blood pressure (a systolic blood pressure, a diastolic blood pressure, and a mean blood pressure) of the subject. The blood pressure sensor is, for example, a blood pressure monitor or a catheter for measuring a vital blood pressure value. The respiration sensor is configured to measure a respiration rate or the like of the subject. The respiration sensor is, for example, an electrode for measuring a respiration rate. The SpO2 sensor is configured to measure the transcutaneous oxygen saturation (SpO2) of the subject. The SpO2 sensor is, for example, a probe for measuring SpO2. The body temperature sensor is configured to measure a body temperature of the subject. The body temperature sensor is a body temperature meter for measuring the body temperature of the subject, a body temperature probe connected to the body temperature meter, or the like. The heart rate sensor is configured to measure the heart rate of the subject. The heart rate sensor is, for example, an electrode for measuring a heart rate. In the present embodiment, the sensor 10 is configured to measure the physiological information by a passive method, but may be configured to measure the physiological information by an active method. In the measurement of physiological information by the passive method, for example, physiological information is measured by receiving a signal from an object without transmitting a signal such as light or electromagnetic waves from the sensor 10 to the object. In the measurement of physiological information by the active method, for example, a signal is transmitted from the sensor 10 to an object, and the physiological information is measured by receiving a signal reflected by the object or transmitted through the object. The physiological information measured by the sensor 10 is input to the information processing device 20 as an electric signal.

The information processing device 20 is, for example, a bed-side monitor. The information processing device 20 can include an input interface 21, a storage 22, a controller 23, and a display unit 24. These components are communicably connected to each other via a bus 25.

The input interface 21 is configured to be input the physiological information on the subject measured by the sensor 10. That is, the physiological information on the subject measured by the sensor 10 is input to the input interface 21. In a case where the physiological information is input to the input interface 21, the input interface 21 generates date and time information indicating a date and time when the physiological information is input to the input interface 21. In the present embodiment, the date and time information is used as information indicating the measurement date and time of the physiological information. For example, in a case where the sensor 10 and the information processing device 20 are electrically connected via a cable, the input interface 21 specifies a type of the sensor 10 according to a type of a connection port of the information processing device 20 to which the cable is connected, and generates the type information on the sensor 10. The input interface 21 associates the physiological information, the date and time information, and the type information, and then transmits these pieces of information to the controller 23.

The storage 22 may include, for example, a storage device (storage) such as a hard disk drive (HDD), a solid state drive (SSD), or a flash memory. The storage 22 is configured to store the physiological information, the date and time information, reference information, name information, change reference information, and the like. The reference information is, for example, information indicating a reference value for determining whether the value indicated by the physiological information is normal. A reference value for determining whether the value indicated by the physiological information is normal is a value defined in a medical guideline or the like, and is, for example, a reference value for determining whether the value indicated by the physiological information is a value indicating that the condition of the subject is normal. For example, in a case where the physiological information is the heart rate, since a normal range of the heart rate is 60 times to 100 times, such reference values are 60 times (a lower limit reference value) and 100 times (an upper limit reference value). For example, when the physiological information is the systolic blood pressure, such reference values are 100 mmHg (the lower limit reference value) and 140 mmHg (the upper limit reference value). For example, when the physiological information is the diastolic blood pressure, such reference values are 60 mmHg (the lower limit reference value) and 90 mmHg (the upper limit reference value). For example, when the physiological information is the mean blood pressure, such reference values are 75 mmHg (the lower limit and upper limit) and 110 mmHg (the upper limit reference value). The name information is information indicating a name of the physiological information on the subject. The name of the physiological information is, for example, the heart rate, the systolic blood pressure, the diastolic blood pressure, the mean blood pressure, a respiration rate, the transcutaneous oxygen saturation, the body temperature, or the like. The change reference information is, for example, information indicating a reference for determining whether there is a change in the physiological information on the subject. The storage 22 is configured to transmit various types of information to the controller 23, for example, according to an instruction signal from the controller 23.

The controller 23 can include a memory 231 and a processor 232, as a hardware configuration. For example, the memory 231 can include a read only memory (ROM) configured to store various computer programs, and a random access memory (RAM) having a plurality of work areas in which various computer programs to be executed by the processor 232 are stored. The processor 232 is, for example, a central processing unit (CPU), and is configured to load a computer program specified from various computer programs incorporated in the ROM on the RAM and execute various processes in cooperation with the RAM.

The computer program may be stored in, for example, various types of non-transitory computer readable medium and supplied to the computer. The non-transitory computer readable medium can include various types of tangible storage media. Examples of the non-transitory computer readable medium include a magnetic recording medium (for example, a flexible disk, a magnetic tape, or a hard disk drive), a magneto-optical recording medium (for example, a magneto-optical disk), a CD-read only memory (ROM), a CD-R, a CD-R/W, and a semi-conductor memory (for example, a mask ROM, a programmable ROM (PROM), an erasable PROM (EPROM), or a flash ROM).

The controller 23 is configured to generate an instruction signal for causing the storage 22 to transmit various types of information stored in the storage 22 to the controller 23. The controller 23 is configured to transmit the generated instruction signal to the storage 22.

The controller 23 is configured to generate the name information, based on the type information received from the input interface 21. The controller 23 is configured to transmit the generated name information to the storage 22.

Based on the physiological information, the date and time information, and the reference information, the controller 23 is configured to generate, for each predetermined unit period, condition information indicating whether the value indicated by the physiological information is a value indicating that the condition of the subject is normal. The predetermined unit period is, for example, an hour, a day, or a month, or the like. For example, in a case where the number of pieces of physiological information indicating values outside the normal range in a certain day is less than a predetermined value, the controller 23 generates condition information indicating that the condition of the subject is good. On the other hand, for example, in a case where the number of pieces of physiological information indicating values outside the normal range in a certain day is equal to or greater than the predetermined value, the controller 23 generates condition information indicating that the condition of the subject is poor. The controller 23 is configured to transmit the generated condition information to the storage 22.

The controller 23 is configured to classify the physiological information input to the input interface 21 into at least a first physiological information group including the physiological information during a first period, a second physiological information group including the physiological information during a second period, and a third physiological information group including the physiological information during a third period. In the present embodiment, the second period is a period different from the first period and is a period later in time than the first period. The third period is a period different from the first period and the second period and is a period later in time than the second period. At least two of the first period, the second period, and the third period may have overlapping periods.

Also in the present embodiment, it is assumed that the current time is 0:00 on Mar. 4, 2024. For example, in a case where the first period is a period from 0:00 on Mar. 2, 2024 to 23:59 on the same day, the second period is a period from 0:00 on Mar. 3, 2024 to 23:59 on the same day, and the third period is a period from 0:00 on Mar. 4, 2024 to 23:59 on the same day, the controller 23 classifies the physiological information during the period from 0:00 on Mar. 2, 2024 to 23:59 on Mar. 4, 2024 into a first physiological information group including physiological information on Mar. 2, 2024, a second physiological information group including physiological information on Mar. 3, 2024, and a third physiological information group including physiological information on Mar. 4, 2024. As described above, in the present embodiment, 24 hours (one day) immediately before the current time is set as the third period, 24 hours (one day) immediately before the third period is set as the second period, and 24 hours (one day) immediately before the second period is set as the first period. The first period, the second period, and the third period may have the same length or different lengths. The first period, the second period, and the third period are not limited to day units, and may be, for example, month units or predetermined time units.

The controller 23 is configured to generate a plurality of pieces of change tendency information indicating a change tendency of the physiological information on the subject from one period to another period, based on the physiological information on the subject input to the input interface 21. In the present embodiment, the controller 23 is configured to determine whether there is a change in the physiological information on the subject, based on the physiological information and the change reference information, and is configured to generate a plurality of pieces of change tendency information based on the determination and the physiological information.

The controller 23 is configured to generate initial change tendency information (an example of the change tendency information) based on, for example, all the physiological information included in the first physiological information group or some of the physiological information included in the first physiological information group. Some of the physiological information included in the first physiological information group is, for example, physiological information input to the input interface 21 for a predetermined time (for example, 30 minutes) from a time (an input start time) when the physiological information on the subject starts to be input to the input interface 21. The initial change tendency information is change tendency information indicating a change tendency of the physiological information on the subject from one period to the other period on the day when the physiological information on the subject starts to be input. For example, the controller 23 is configured to generate first change tendency information (an example of change tendency information) indicating a change tendency of the physiological information on the subject from the first period to the second period, based on the first physiological information group and the second physiological information group. For example, the controller 23 is configured to generate second change tendency information (an example of change tendency information) indicating a change tendency of the physiological information on the subject from the second period to the third period, based on the second physiological information group and the third physiological information group.

The initial change tendency information is generated based on, for example, a difference between a value indicated by the physiological information input to the input interface 21 at the input start time and a value indicated by the physiological information input to the input interface 21 30 minutes after the input start time. The difference is, for example, a value (an initial comparison value) obtained by subtracting the value indicated by the physiological information input to the input interface 21 at the input start time from the value indicated by the physiological information input to the input interface 21 30 minutes after the input start time. Based on the difference and the change reference information, the controller 23 is configured to determine whether the physiological information on the subject changes in 30 minutes from the input start time. The controller 23 is configured to generate the initial change tendency information, based on the determination and the initial comparison value.

The first change tendency information is generated based on a first comparison value calculated from a first value based on the first physiological information group and a second value based on the second physiological information group. The second change tendency information is generated based on a second comparison value calculated from the second value based on the second physiological information group and a third value based on the third physiological information group. Note that, for example, the first value is a total value of values indicated by the pieces of physiological information included in the first physiological information group. The second value is a total value of values indicated by the pieces of physiological information included in the second physiological information group. The third value is a total value of values indicated by the pieces of physiological information included in the third physiological information group. For example, the first comparison value is a difference between the first value and the second value (a value obtained by subtracting the first value from the second value), and the second comparison value is a difference between the second value and the third value (a value obtained by subtracting the second value from the third value). In the present embodiment, the controller 23 is configured to determine whether there is a change in the physiological information on the subject from the first period to the second period, based on the first comparison value and the change reference information. The controller 23 is configured to generate the first change tendency information, based on the determination and the first comparison value. In addition, the controller 23 is configured to determine whether there is a change in the physiological information on the subject from the second period to the third period based on the second comparison value and the change reference information. The controller 23 is configured to generate the second change tendency information, based on the determination and the second comparison value.

The controller 23 is configured to generate display data in which a plurality of pieces of change tendency information are displayed side by side or a plurality of pieces of change tendency information are displayed so as to be overlapped with each other, based on the generated plurality of pieces of change tendency information. For example, in a case where the controller 23 generates the display data based on the initial change tendency information, the first change tendency information, and the second change tendency information, the initial change tendency information, the first change tendency information, and the second change tendency information are displayed side by side or displayed so as to be overlapped with each other in the display data. In the present embodiment, in the display data, the change tendency information is displayed as a figure having an inclination. In the present specification, an arrow-shaped figure is used as an example of a figure having an inclination, but the figure having an inclination is not limited to the arrow-shaped figure. Further, in the present specification, a rightward horizontal arrow-shaped figure (see a first figure D1 in FIG. 3) is set as a reference figure, and a direction (horizontal direction) in which the reference figure is facing is set as a reference direction. The “inclination” in the present specification means an inclination with respect to the reference direction. Therefore, the inclination of the rightward horizontal arrow-shaped figure is zero. In this specification, a figure having a zero inclination is also included in the figure having an inclination. In the present embodiment, the controller 23 generates display data including the name information. The controller 23 is configured to transmit the generated display data to the display unit 24. A display mode of the initial change tendency information changes according to the initial comparison value. A display mode of the first change tendency information changes according to the first comparison value. A display mode of the second change tendency information changes according to the second comparison value. For example, in a case where the initial change tendency information is displayed as an arrow-shaped figure in the display data, the inclination of the arrow-shaped figure corresponding to the initial change tendency information changes according to the initial comparison value. For example, in a case where the first change tendency information is displayed as an arrow-shaped figure in the display data, the inclination of the arrow-shaped figure corresponding to the first change tendency information changes according to the first comparison value. For example, in a case where the second change tendency information is displayed as an arrow-shaped figure in the display data, the inclination of the arrow-shaped figure corresponding to the second change tendency information changes according to the second comparison value. That is, the change in the display mode here refers to a change in the inclination of the arrow-shaped figure.

The display modes of the arrow-shaped figures corresponding to the initial change tendency information, the first change tendency information, and the second change tendency information are determined based on the condition information. For example, in a case where the condition information indicates that the condition of the subject is good, the arrow-shaped figure is colored with light red, whereas in a case where the condition information indicates that the condition of the subject is poor, the arrow-shaped figure is colored with dark red. That is, the display mode of the arrow-shaped figure referred to here indicates the shading of the color of the arrow-shaped figure.

The display unit 24 is, for example, a display such as a liquid crystal display or an organic EL display. The display unit 24 is configured to display an image based on the display data received from the controller 23, for example.

### First Example of First Embodiment

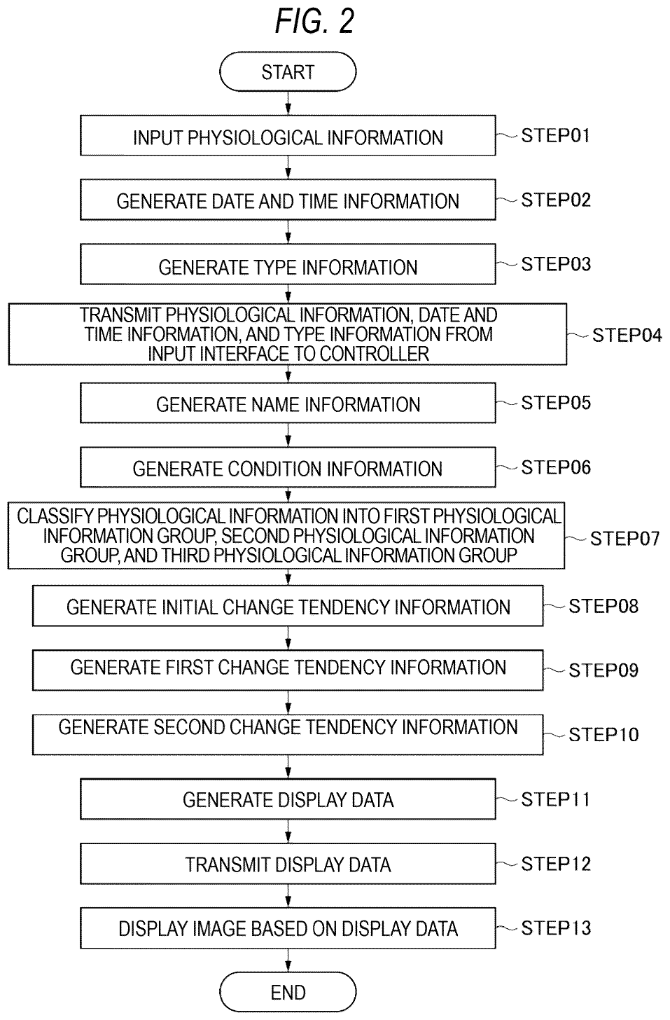

Next, an information processing method according to a first example of the first embodiment will be described with reference to FIGS. 2 and 3. FIG. 2 is a flowchart illustrating an example of the information processing method until an image 100 based on the display data is displayed on the display unit 24 of the information processing device 20. FIG. 3 illustrates the image 100 based on the display data. Note that the premise in the present example described below is the same or similar in the following examples unless otherwise specified.

The physiological information in the present example is a heart rate. Therefore, in the present example, “physiological information” refers to a heart rate unless otherwise specified. In the present example, it is assumed that the sensor 10 is configured to measure physiological information on the heart rate every minute. In the present example, the sensor 10 and the information processing device 20 are electrically connected via a cable, and the input interface 21 is configured to specify the type of the sensor 10 according to the type of the connection port of the information processing device 20 to which the cable is connected, and is configured to generate the type information on the sensor 10.

In the present example, the controller 23 is configured to classify the physiological information input to the input interface 21 into the first physiological information group including the physiological information during the first period, the second physiological information group including the physiological information during the second period, and the third physiological information group including the physiological information during the third period. It is assumed that the first period is a period from 0:00 on Mar. 2, 2024 to 23:59 on the same day, the second period is a period from 0:00 on Mar. 3, 2024 to 23:59 on the same day, and the third period is a period from 0:00 on Mar. 4, 2024 to 23:59 on the same day. As described above, in the present example, the first period, the second period, and the third period have the same length.

In the present example, it is assumed that the controller 23 is configured to generate the initial change tendency information, based on the physiological information input to the input interface 21 from 0:00 on Mar. 2, 2024 to 0:30 on the same day. However, the controller 23 may be configured to generate the initial change tendency information, based on, for example, the physiological information input to the input interface 21 from 0:00 on Mar. 2, 2024 to 23:59 on the same day. Further, in the present example, it is assumed that both the values indicated by the physiological information input to the input interface 21 at 0:00 on Mar. 2, 2024 and the physiological information input to the input interface 21 at 0:30 on the same day are 60 times.

In the present example, the first value is the total value of the values indicated by the pieces of physiological information included in the first physiological information group, and is 116000 times/day. The second value is the total value of the values indicated by the pieces of physiological information included in the second physiological information group, and is 135000 times/day. The third value is the total value of the values indicated by the pieces of physiological information included in the third physiological information group, and is 158000 times/day.

In the present example, the change tendency information is displayed as an arrow-shaped figure having an inclination. In a case where a value obtained by subtracting the value indicated by the physiological information at a time 30 minutes after the input start time from the value indicated by the physiological information at the input start time is less than −10, the controller 23 tilts the arrow-shaped figure corresponding to the initial change tendency information downward from the reference direction according to the value. In a case where the value obtained by subtracting the value indicated by the physiological information at the time 30 minutes after the input start time from the value indicated by the physiological information at the input start time is −10 or more and less than +10, the controller 23 sets the inclination of the arrow-shaped figure corresponding to the initial change tendency information to zero (sets the figure horizontal to the reference direction) according to the value. In a case where the value obtained by subtracting the value indicated by the physiological information at the time 30 minutes after the input start time from the value indicated by the physiological information at the input start time is +10 or more, the controller 23 tilts the arrow-shaped figure corresponding to the initial change tendency information upward from the reference direction according to the value. In a case where a value obtained by subtracting the total value of the values indicated by the pieces of physiological information on the previous day of a certain day from the total value of the values indicated by the pieces of physiological information on the certain day is −15000, the controller 23 tilts the arrow-shaped figure corresponding to the first change tendency information or the second change tendency information downward from the reference direction according to the value. In a case where the value obtained by subtracting the total value of the values indicated by the pieces of physiological information on the previous day of a certain day from the total value of the values indicated by the pieces of physiological information on the certain day is −15000 or more and less than +15000, the controller 23 sets the inclination of the arrow-shaped figure corresponding to the first change tendency information or the second change tendency information to zero (sets the figure horizontal to the reference direction). In a case where the value obtained by subtracting the total value of the values indicated by the pieces of physiological information on the previous day of a certain day from the total value of the values indicated by the pieces of physiological information on the certain day is equal to or greater than +15000, the controller 23 tilts the arrow-shaped figure corresponding to the first change tendency information or the second change tendency information upward from the reference direction according to the value.

In the present example, the condition information is generated on a daily basis, and the predetermined value related to the number of pieces of physiological information indicating the values outside the normal range in a certain day is 100. In the present example, it is assumed that the number of pieces of physiological information indicating the values outside the normal range on Mar. 2, 2024 is 0. It is assumed that the number of pieces of physiological information indicating the values outside the normal range on Mar. 3, 2024 is 50. It is assumed that the number of pieces of physiological information indicating the values outside the normal range on Mar. 4, 2024 is 200. In a case where the condition information indicates that the condition of the subject is good, the arrow-shaped figure corresponding to the change tendency information during the period corresponding to the condition information is colored with light red, whereas in a case where the condition information indicates that the condition of the subject is poor, the arrow-shaped figure corresponding to the change tendency information during the period corresponding to the condition information is colored with dark red.

As illustrated in FIG. 2, the physiological information measured by the sensor 10 is input to the input interface 21 of the information processing device 20 (STEP01). In the present example, the physiological information measured by the sensor 10 during the period from 0:00 on Mar. 2, 2024 to 23:59 on Mar. 4, 2024 is sequentially input to the input interface 21 every processing unit time as time elapses. The processing unit time is, for example, one hour, one minute, or one second.

When the physiological information is input to the input interface 21, the input interface 21 generates the date and time information indicating the date and time when the physiological information is input to the input interface 21 (STEP02).

The input interface 21 specifies the type of the sensor 10 according to the type of the connection port of the information processing device 20 to which the cable connecting the sensor 10 and the information processing device 20 is connected, and generates the type information on the sensor 10 (STEP03). In the present example, since the physiological information input to the input interface 21 is the heart rate, the input interface 21 generates type information indicating that the sensor 10 is a heart rate sensor.

The input interface 21 associates the physiological information, the date and time information, and the type information, and then transmits these pieces of information to the controller 23 (STEP04).

The controller 23 generates the name information based on the type information received from the input interface 21 (STEP05). In the present example, since the physiological information input to the input interface 21 is the heart rate, the controller 23 generates the name information indicating that the name of the physiological information is the heart rate.

The controller 23 generates the condition information on a daily basis, based on the physiological information and the reference information (STEP06). In the present example, since the physiological information input to the input interface 21 is the heart rate, the controller 23 determines whether the value indicated by the physiological information is within a range of 60 times to 100 times (a normal range of the heart rate). In the present example, the number (0) of pieces of physiological information indicating values outside the normal range on Mar. 2, 2024 and the number (50) of pieces of physiological information indicating values outside the normal range on Mar. 3, 2024 are less than the predetermined value (100). On the other hand, the number (200) of pieces of physiological information indicating values outside the normal range on Mar. 4, 2024 is equal to or greater than the predetermined value (100). Therefore, the controller 23 generates the condition information indicating that the condition of the subject is good on Mar. 2, 2024 and Mar. 3, 2024 and the condition information indicating that the condition of the subject is poor on Mar. 4, 2024.

The controller 23 classifies the physiological information input to the input interface 21 into the first physiological information group including the physiological information during the first period, the second physiological information group including the physiological information during the second period, and the third physiological information group including the physiological information during the third period (STEP07). In the present example, the controller 23 classifies the physiological information input to the input interface 21 into the first physiological information group including the physiological information on Mar. 2, 2024, the second physiological information group including the physiological information on Mar. 3, 2024, and the third physiological information group including the physiological information on Mar. 4, 2024.

The controller 23 generates the initial change tendency information, based on all the physiological information included in the first physiological information group or some of the physiological information included in the first physiological information group (STEP08). In the present example, the controller 23 generates the initial change tendency information, based on the physiological information input to the input interface 21 at 0:00 on Mar. 2, 2024 and the physiological information input to the input interface 21 at 0:30 on the same day. Further, in the present example, both the values indicated by the physiological information (the heart rate) input to the input interface 21 at 0:00 on Mar. 2, 2024 and the physiological information input to the input interface 21 at 0:30 on the same day are 60 times. Therefore, the value obtained by subtracting the value indicated by the physiological information input on at 0:00 on Mar. 2, 2024 from the value indicated by the physiological information input to the input interface 21 at 0:30 on Mar. 2, 2024 is 0 (that is, −10 or more and less than +10). For this reason, the controller 23 generates the initial change tendency information indicating that the change tendency of the physiological information on the subject from 0:00 on Mar. 2, 2024 to 0:30 on the same day is flat.

The controller 23 generates the first change tendency information indicating a change tendency of the physiological information on the subject from the first period to the second period, based on the first physiological information group and the second physiological information group (STEP09). More specifically, the controller 23 generates the first change tendency information, based on the first comparison value calculated from the first value based on the first physiological information group and the second value based on the second physiological information group. In the present example, since the first value is 116000 times/day and the second value is 135000 times/day, the first comparison value is +19000. That is, since the first comparison value is +15000 or more, the controller 23 tilts the arrow-shaped figure upward from the reference direction according to the first comparison value. Therefore, the controller 23 generates the first change tendency information indicating that the change tendency of the physiological information on the subject from Mar. 2, 2024 to Mar. 3, 2024 is an upward tendency.

The controller 23 generates the second change tendency information indicating a change tendency of the physiological information on the subject from the second period to the third period, based on the second physiological information group and the third physiological information group (STEP10). More specifically, the controller 23 generates the second change tendency information, based on the second comparison value calculated from the second value based on the second physiological information group and the third value based on the third physiological information group. In the present example, since the second value is 135000 times/day and the third value is 158000 times/day, the second comparison value is +23000. That is, since the second comparison value is +15000 or more, the controller 23 tilts the arrow-shaped figure upward from the reference direction according to the second comparison value. Therefore, the controller 23 generates the second change tendency information indicating that the change tendency of the physiological information on the subject from Mar. 3, 2024 to Mar. 4, 2024 is an upward tendency. Since the second comparison value is greater than the first comparison value, the inclination of the arrow-shaped figure corresponding to the second change tendency information is greater than the inclination of the arrow-shaped figure corresponding to the first change tendency information.

The controller 23 generates the display data, based on the name information, the condition information, the initial change tendency information, the first change tendency information, and the second change tendency information (STEP11). In the present example, the display data includes the physiological information and the name information.

The controller 23 transmits the generated display data to the display unit 24 (STEP12). In a case where the display data is transmitted to the display unit 24, the image 100 illustrated in FIG. 3 is displayed on the display unit 24 (STEP13).

Here, the image 100 based on the display data displayed by the display unit 24 will be described with reference to FIG. 3. As illustrated in FIG. 3, the image 100 can include a first name M1, a first axis AX1, a second axis AX2, a heart rate graph object G1, a first figure D1, a second figure D2, and a third figure D3. For example, when the heart rate is 60 times, the value related to the physiological information is 60 times (in this example, the heart rate). That is, the value related to the physiological information is a magnitude, a degree, or the like indicated by the physiological information.

The first name M1 is based on the name information on the heart rate. Therefore, the first name M1 is the heart rate. The first name M1 is displayed on an upper left side of the display unit 24. The first axis AX1 is a timeline and extends in a lateral direction (the left-right direction in FIG. 3). The second axis AX2 indicates a value related to the physiological information (the heart rate) and extends in a vertical direction (the up-down direction in FIG. 3).

The heart rate graph object G1 corresponds to physiological information on the heart rate. The heart rate graph object G1 indicates the magnitude of each heart rate. The heart rate graph object G1 is displayed in a first region R1 defined by the first axis AX1 and the second axis AX2. In a left region R11 located on a left side of the first region R1, a graph object corresponding to the physiological information on the heart rate on Mar. 2, 2024, in the heart rate graph object G1, is displayed. In a central region R12 located at a center of the first region R1, a graph object corresponding to the physiological information on the heart rate on Mar. 3, 2024, in the heart rate graph object G1, is displayed. In a right region R13 located on a right side of the first region R1, a graph object corresponding to the physiological information on the heart rate on Mar. 4, 2024, in the heart rate graph object G1, is displayed.

The first figure D1 corresponds to the initial change tendency information. The second figure D2 corresponds to the first change tendency information. The third figure D3 corresponds to the second change tendency information. The first figure D1, the second figure D2, and the third figure D3 are displayed above the graph object corresponding to the physiological information on the heart rate on Mar. 4, 2024 in the heart rate graph object G1. That is, the first figure D1, the second figure D2, and the third figure D3 are displayed on an upper right side of the display unit 24. However, the display positions of the first figure D1, the second figure D2, and the third figure D3 are not limited to this example. For example, the first figure D1, the second figure D2, and the third figure D3 may be displayed on the upper left side or the upper center of the display unit 24. The first figure D1, the second figure D2, and the third figure D3 are displayed side by side, and an arrangement order of the first figure D1, the second figure D2, and the third figure D3 indicates a temporal order. That is, the plurality of pieces of change tendency information in the display data are displayed side by side, and the arrangement order of the plurality of pieces of change tendency information indicates the temporal order of the change tendency information.

The first figure D1, the second figure D2, and the third figure D3 are figures each having an inclination. The first figure D1, the second figure D2, and the third figure D3 are displayed in an arrow shape. Since the initial change tendency information indicates that the change tendency of the physiological information on the subject from 0:00 on Mar. 2, 2024 to 0:30 on the same day is flat, the inclination of the first figure D1 is zero (that is, horizontal with respect to the reference direction). On the other hand, since the first change tendency information indicates that the change tendency of the physiological information on the subject from Mar. 2, 2024 to Mar. 3, 2024 is an upward tendency, the second figure D2 is inclined upward from the reference direction. In addition, since the second change tendency information indicates that the change tendency of the physiological information on the subject from Mar. 3, 2024 to Mar. 4, 2024 is an upward tendency, the third figure D3 is inclined upward from the reference direction. Further, since the second comparison value is greater than the first comparison value, the inclination of the third figure D3 is greater than the inclination of the second figure D2. As described above, the inclination of each figure corresponding to each piece of change tendency information indicates an amount of change in the physiological information on the subject from one period to the other period.

In the present example, the display modes of the first figure D1, the second figure D2, and the third figure D3 (the shading of the colors of the first figure D1, the second figure D2, and the third figure D3) are determined based on the condition information. In the present example, the condition information on Mar. 2, 2024 and Mar. 3, 2024 indicates that the condition of the subject is good, whereas the condition information on Mar. 4, 2024 indicates that the condition of the subject is poor. Therefore, while the first figure D1 and the first second figure D2 are colored with light red, the third figure D3 is colored with dark red. In FIG. 3, the number of hatched lines represents the shading of color. Thus, the display modes of the first figure D1, the second figure D2, and the third figure D3 are determined based on the physiological information and the reference information.

In order to accurately recognize a condition of the subject, it is important to grasp not only the physiological information on the subject but also a change tendency of the physiological information on the subject, but it may be difficult to immediately grasp the change tendency only by glancing the physiological information on the subject.

According to the information processing device 20 having the above configuration, the information processing device 20 is configured to generate a plurality of pieces of change tendency information indicating a change tendency of the physiological information on the subject during one period to the other period based on the physiological information on the subject. Then, the information processing device 20 is configured to generate display data in which the plurality of pieces of generated change tendency information are displayed side by side. A user such as a medical worker can intuitively and accurately grasp the change tendency of the physiological information on the subject by visually recognizing the image 100 based on such display data, for example. Therefore, according to the information processing device 20, it is possible to support the user to intuitively and accurately grasp the change tendency of the physiological information on the subject. In addition, the information processing method, the computer program and the non-transitory computer readable medium according to the above configurations can also achieve the same or similar effects.

According to the information processing device 20 having the above configuration, in the display data, the change tendency information is displayed as a figure having an inclination, and the inclination indicates the amount of change in the physiological information from one period to the other period. The display mode of the figure is determined based on the physiological information and the reference information indicating a reference value for determining whether the value indicated by the physiological information is normal. Therefore, the user can immediately grasp the amount of change of the physiological information and whether the value indicated by the physiological information is normal by using the display data generated by the information processing device 20.

According to the information processing device 20 having the above configuration, the plurality of pieces of change tendency information in the display data are displayed side by side, and the arrangement order of the plurality of pieces of change tendency information indicates the temporal order of the change tendency information. Therefore, for example, the user can grasp the change tendency of the physiological information on the subject in time series by visually recognizing the image 100 based on such display data.

According to the information processing device 20 having the above configuration, the information processing device 20 is configured to generate the first change tendency information indicating the change tendency of the physiological information on the subject from the first period to the second period based on the first physiological information group and the second physiological information group. The information processing device 20 is configured to generate the second change tendency information indicating a change tendency of the physiological information on the subject from the second period to the third period based on the second physiological information group and the third physiological information group. In the display data, the first change tendency information and the second change tendency information are displayed side by side. For example, by visually recognizing the image 100 based on such display data, the user can intuitively and accurately grasp the change tendency of the physiological information on the subject from the first period to the second period and the change tendency of the physiological information on the subject from the second period to the third period. Therefore, according to the information processing device 20, it is possible to support the user to intuitively and accurately grasp the change tendency of the physiological information on the subject over a plurality of periods.

According to the information processing device 20 having the above configuration, the first change tendency information is generated based on the first comparison value calculated from the first value and the second value, and the second change tendency information is generated based on the second comparison value calculated from the second value and the third value. As described above, since the first change tendency information and the second change tendency information are calculated from the first comparison value or the second comparison value, which is objective information, the user can grasp the change tendency of the physiological information on the subject based on the objective information.

According to the information processing device 20 having the above configuration, since the first comparison value is the difference between the first value and the second value and the second comparison value is the difference between the second value and the third value, the first change tendency information and the second change tendency information can be generated by relatively simple information processing.

According to the information processing device 20 having the above configuration, the display mode of the first change tendency information changes according to the first comparison value, and the display mode of the second change tendency information changes according to the second comparison value. Therefore, for example, the user can intuitively and easily grasp the degree of the change tendency of the physiological information on the subject by visually recognizing the image 100 based on such display data.

According to the information processing device 20 having the above configuration, the display data includes the physiological information. Therefore, for example, by visually recognizing the image 100 based on such display data, the user can grasp the physiological information (the graph object corresponding to the physiological information) together with the change tendency of the physiological information on the subject. Therefore, for example, the user can more accurately grasp the change tendency of the physiological information on the subject by visually recognizing the image 100 based on such display data.

According to the information processing device 20 having the above configuration, since the change tendency information is displayed in an arrow shape in the display data, the user can intuitively and easily recognize the change tendency of the physiological information on the subject.

According to the information processing device 20 having the above configuration, since the display data includes the name information indicating the name of the physiological information on the subject, the user can recognize which physiological information the change tendency relates to and then grasp the change tendency, for example, by visually recognizing the image 100 based on such display data.

### Second Example of First Embodiment

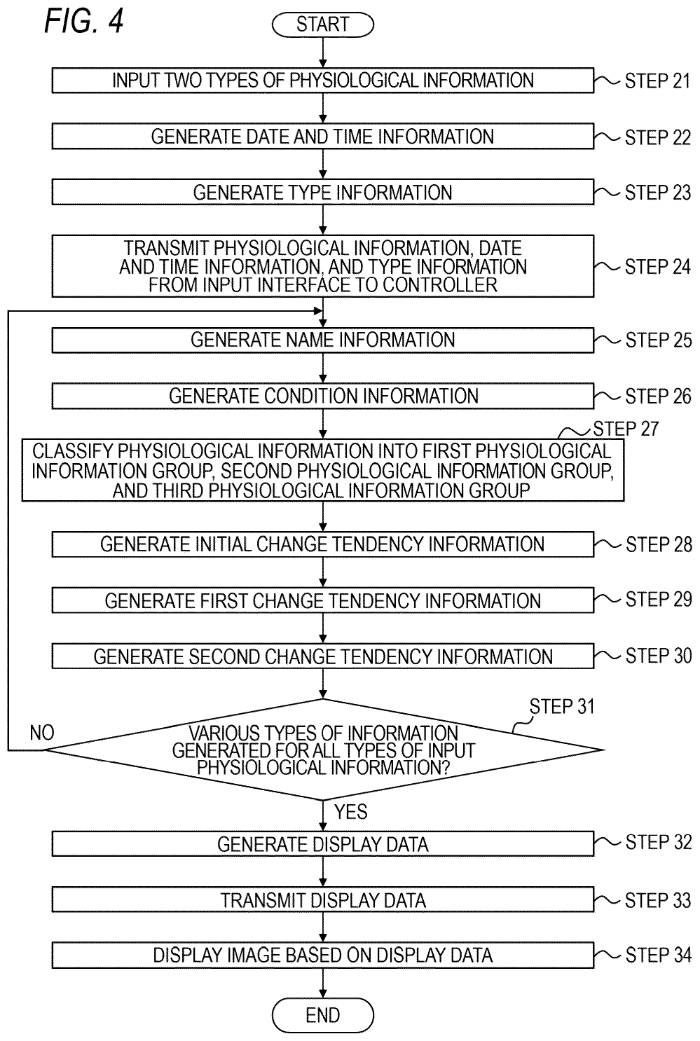

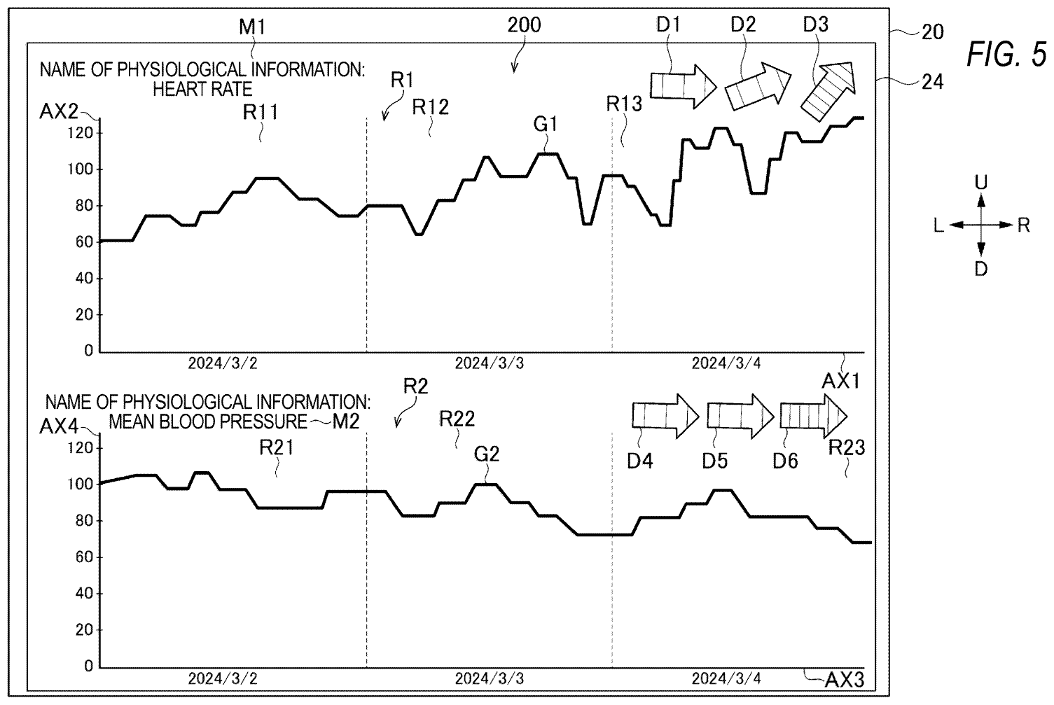

Next, an information processing method according to a second example of the first embodiment will be described with reference to FIGS. 4 and 5. FIG. 4 is a flowchart illustrating an example of the information processing method until an image 200 based on the display data is displayed on the display unit 24 of the information processing device 20. FIG. 5 illustrates the image 200 based on the display data. In the second example, parts the same or similar to those of the first example of the first embodiment are denoted by the same or similar reference numerals as those of the first example of the first embodiment, and description of repeated parts is omitted as appropriate. The present example is different from the first example of the first embodiment in that the controller 23 executes the information processing method illustrated in FIG. 4 instead of the information processing method illustrated in FIG. 2.

In the present example, two types of physiological information measured by the sensor 10 is input to the input interface 21. In the present example, the physiological information input to the input interface 21 is a heart rate and a mean blood pressure. However, the physiological information input to the input interface 21 may be, for example, a heart rate and a diastolic blood pressure, or may be a heart rate and a systolic blood pressure. In the present example, the sensor 10 is configured to measure the heart rate every minute and the mean blood pressure every 30 minutes. The input interface 21 is configured to transmit the two types of input physiological information to the controller 23. Also in the present example, the input interface 21 is configured to generate the date and time information and the type information on the sensor 10. However, in the present example, the input interface 21 is configured to generate the date and time information and the type information on the sensor 10 for each type of physiological information. In addition, the input interface 21 is configured to associate the physiological information, the date and time information, and the type information with each other for each type of physiological information, and then transmit these pieces of information to the controller 23.

In the present example, the controller 23 is configured to generate a plurality of pieces of change tendency information for each type of physiological information, based on the two types of physiological information. Also in the present example, the controller 23 is configured to generate the display data. However, in the present example, in the display data generated by the controller 23, the plurality of pieces of change tendency information are displayed side by side for each type of physiological information.

In the present example, the two types of physiological information from 0:00 on Mar. 2, 2024 to 0:30 on the same day are input to the input interface 21. The various kinds of information on the heart rate in the present example are the same as or similar to the various kinds of information on the heart rate in the first example of the first embodiment. On the other hand, various types of information on the mean blood pressure will be described later.

In the present example, it is assumed that the value indicated by the mean blood pressure input to the input interface 21 at 0:00 on Mar. 2, 2024 is 105 mmHg, and the value indicated by the mean blood pressure input to the input interface 21 at 0:30 on the same day is 100 mmHg.

In the present example, it is assumed that the first value related to the mean blood pressure is the total value of the values indicated by the pieces of physiological information included in the first physiological information group, and is 4600 mmHg/day. It is assumed that the second value related to the mean blood pressure is the total value of the values indicated by the pieces of physiological information included in the second physiological information group, and is 4300 mmHg/day. It is assumed that the third value related to the mean blood pressure is the total value of the values indicated by the pieces of physiological information included in the third physiological information group, and is 3850 mmHg/day.

In the present example, the change tendency information on the mean blood pressure is displayed as an arrow-shaped figure having an inclination. In a case where the value obtained by subtracting a mean blood pressure at the time 30 minutes after the input start time from a mean blood pressure at the input start time is less than −10, the controller 23 tilts the arrow-shaped figure corresponding to the initial change tendency information downward from the reference direction according to the value. In a case where the value obtained by subtracting the mean blood pressure at the time 30 minutes after the input start time from the mean blood pressure at the input start time is −10 or more and less than +10, the controller 23 sets the inclination of the arrow-shaped figure corresponding to the initial change tendency information to zero (sets the figure horizontal to the reference direction) according to the value. In a case where the value obtained by subtracting the mean blood pressure at the time 30 minutes after the input start time from the mean blood pressure at the input start time is +10 or more, the controller 23 tilts the arrow-shaped figure corresponding to the initial change tendency information upward from the reference direction according to the value. In a case where the value obtained by subtracting the total value of the mean blood pressures on the previous day of a certain day from the total value of the mean blood pressures on the certain day is less than −500, the controller 23 tilts the arrow-shaped figure corresponding to the first change tendency information or the second change tendency information downward from the reference direction according to the value. In a case where the value obtained by subtracting the total value of the mean blood pressures on the previous day of a certain day from the total value of the mean blood pressures on the certain day is −500 or more and less than +500, the controller 23 sets the inclination of the arrow-shaped figure corresponding to the first change tendency information or the second change tendency information to zero (sets the figure horizontal to the reference direction). In a case where the value obtained by subtracting the total value of the mean blood pressures on the previous day of a certain day from the total value of the mean blood pressures on the certain day is +500 or more, the controller 23 tilts the arrow-shaped figure corresponding to the first change tendency information or the second change tendency information upward from the reference direction according to the value.

In the present example, the condition information on the mean blood pressure is generated on a daily basis, and the predetermined value related to the number of mean blood pressures indicating values outside a normal range in a certain day is 24. In the present example, it is assumed that the number of mean blood pressures indicating the values outside the normal range on Mar. 2, 2024, the number of mean blood pressures indicating the values outside the normal range on Mar. 3, 2024, and the number of mean blood pressures indicating the values outside the normal range on Mar. 4, 2024 are all 0. Regarding the mean blood pressure, when the condition information indicates that the condition of the subject is good, the arrow-shaped figure corresponding to the change tendency information during the period corresponding to the condition information is colored with light red, whereas when the condition information indicates that the condition of the subject is poor, the arrow-shaped figure corresponding to the change tendency information during the period corresponding to the condition information is colored with dark red.

As illustrated in FIG. 4, the two types of physiological information (the heart rate and the mean blood pressure) measured by the sensor 10 are input to the input interface 21 of the information processing device 20 (STEP21). In the present example, the two types of physiological information measured by the sensor 10 during the period from 0:00 on Mar. 2, 2024 to 23:59 on Mar. 4, 2024 are sequentially input to the input interface 21 every processing unit time as time elapses.

STEP22 to STEP30 are the same as or similar to STEP02 to STEP10 in the first example of the first embodiment. However, in the present example, the input interface 21 generates not only the date and time information and the type information on the heart rate but also these pieces of information on the mean blood pressure (STEP22 and STEP23). The input interface 21 associates the physiological information, the date and time information, and the type information on the heart rate and the mean blood pressure, and then transmits these pieces of information to the controller 23 (STEP24). In the present example, the controller 23 generates not only the name information and the condition information on the heart rate but also these pieces of information on the mean blood pressure (STEP25 and STEP26). The controller 23 classifies not only the physiological information on the heart rate but also the physiological information on the mean blood pressure into the first physiological information group, the second physiological information group, and the third physiological information group (STEP27). The controller 23 generates not only the initial change tendency information, the first change tendency information, and the second change tendency information on the heart rate but also these pieces of information on the mean blood pressure (STEP28 to STEP30).

Here, the generation of the condition information, the initial change tendency information, the first change tendency information, and the second change tendency information on the mean blood pressure in the present example will be described in detail. In the present example, the number of mean blood pressures indicating the values outside the normal range from Mar. 2, 2024 to Mar. 4, 2024 is 0, which is less than the predetermined value (24). Therefore, the controller 23 generates the condition information indicating that the condition of the subject is good on the mean blood pressure from Mar. 2, 2024 to Mar. 4, 2024 (STEP26).

In the present example, the mean blood pressure input to the input interface 21 at 0:00 on Mar. 2, 2024 is 105 mmHg, and the mean blood pressure input to the input interface 21 at 0:30 on the same day is 100 mmHg. Therefore, the value obtained by subtracting the mean blood pressure input at 0:00 on Mar. 2, 2024 from the mean blood pressure input to the input interface 21 at 0:30 on Mar. 2, 2024 is −5 (that is, −10 or more and less than +10). For this reason, the controller 23 generates the initial change tendency information indicating that the change tendency of the mean blood pressure from 0:00 on Mar. 2, 2024 to 0:30 on the same day is flat (STEP28).

In the present example, since the first value is 4600 mmHg/day and the second value is 4300 mmHg/day, the first comparison value is −300. That is, since the first comparison value is-500 or more and less than +500, the controller 23 generates the first change tendency information indicating that the change tendency of the mean blood pressure from the first period to the second period is flat (STEP29).

In the present example, since the second value is 4300 mmHg/day and the third value is 3850 mmHg/day, the second comparison value is −450. That is, since the second comparison value is −500 or more and less than +500, the controller 23 generates the second change tendency information indicating that the change tendency of the mean blood pressure from the second period to the third period is flat (STEP30).

Next, STEP31 will be described. The controller 23 determines whether various types of information such as change tendency information have been generated for all types of physiological information input to the input interface 21 (STEP31). When it is determined that various types of information have been generated for all types of physiological information input to the input interface 21 (YES in STEP31), the controller 23 executes STEP32. On the other hand, when it is determined that there is physiological information for which various types of information have not been generated among the physiological information input to the input interface 21 (NO in STEP31), the controller 23 executes STEP25 again for the physiological information for which various types of information have not been generated. In the present example, when various types of information such as the change tendency information on the heart rate and the mean blood pressure are generated, the controller 23 determines that various types of information have been generated for all types of physiological information input to the input interface 21 (YES in STEP31), and executes STEP32.

STEP32 to STEP34 are the same as or similar to STEP11 to STEP13 in the first example of the first embodiment. However, in the present example, the image 200 illustrated in FIG. 5 is displayed on the display unit 24.

Here, the image 200 based on the display data displayed in the display unit 24 will be described with reference to FIG. 5. As illustrated in FIG. 5, the image 200 can include the first name M1, the second name M2, the first axis AX1, the second axis AX2, a third axis AX3, a fourth axis AX4, the heart rate graph object G1, a mean blood pressure graph object G2, the first figure D1, the second figure D2, the third figure D3, a fourth figure D4, a fifth figure D5, and a sixth figure D6.

The second name M2 is based on the name information on the mean blood pressure. Therefore, in the present example, the second name M2 is the mean blood pressure. The second name M2 is displayed at the left center of the display unit 24. The third axis AX3 is a timeline and extends in the lateral direction (the left-right direction in FIG. 5). The fourth axis AX4 indicates a value related to the mean blood pressure and extends in the vertical direction (the up-down direction in FIG. 5).

The mean blood pressure graph object G2 corresponds to physiological information on the mean blood pressure. The mean blood pressure graph object G2 indicates the magnitude of each mean blood pressure. The mean blood pressure graph object G2 is displayed in a second region R2 defined by the third axis AX3 and the fourth axis AX4. In a left region R21 located on a left side of the second region R2, a graph object corresponding to the physiological information on the mean blood pressure on Mar. 2, 2024, in the mean blood pressure graph object G2, is displayed. In a central region R22 located at a center of the second region R2, a graph object corresponding to the physiological information on the mean blood pressure on Mar. 3, 2024, in the mean blood pressure graph object G2, is displayed. In a right region R23 located on a right side of the second region R2, a graph object corresponding to the physiological information on the mean blood pressure on Mar. 4, 2024, in the mean blood pressure graph object G2, is displayed.

The fourth figure D4 corresponds to the initial change tendency information on the mean blood pressure. The fifth figure D5 corresponds to the first change tendency information on the mean blood pressure. The sixth figure D6 corresponds to the second change tendency information on the mean blood pressure. The fourth figure D4, the fifth figure D5, and the sixth figure D6 are displayed at the right center of the display unit 24. However, the display positions of the fourth figure D4, the fifth figure D5, and the sixth figure D6 are not limited to this example. For example, the fourth figure D4, the fifth figure D5, and the sixth figure D6 may be displayed at the left center or the upper right side of the display unit 24. The fourth figure D4, the fifth figure D5, and the sixth figure D6 are displayed side by side, and an arrangement order of the fourth figure D4, the fifth figure D5, and the sixth figure D6 indicates a temporal order. That is, the change tendency information on the plurality of mean blood pressures in the display data is displayed side by side, and the arrangement order of the change tendency information on the plurality of mean blood pressures indicates the temporal order of the change tendency information on the mean blood pressure.

The fourth figure D4, the fifth figure D5, and the sixth figure D6 are figures each having an inclination. The fourth figure D4, the fifth figure D5, and the sixth figure D6 are displayed in an arrow shape. Since the initial change tendency information on the mean blood pressure indicates that the change tendency of the mean blood pressure of the subject from 0:00 on Mar. 2, 2024 to 0:30 on the same day is flat, the inclination of the fourth figure D4 is zero. Since the first change tendency information on the mean blood pressure indicates that the mean blood pressure of the subject from Mar. 2, 2024 to Mar. 3, 2024 is flat, the inclination of the fifth figure D5 is zero. Since the second change tendency information on the mean blood pressure indicates that the change tendency of the mean blood pressure of the subject from Mar. 3, 2024 to Mar. 4, 2024 is flat, the inclination of the sixth figure D6 is zero.

In the present example, the display modes of the fourth figure D4, the fifth figure D5, and the sixth figure D6 (the shading of the colors of the fourth figure D4, the fifth figure D5, and the sixth figure D6) are determined based on the condition information on the mean blood pressure. In the present example, the condition information on the mean blood pressure on Mar. 2, 2024, Mar. 3, 2024 and Mar. 4, 2024 indicates that the condition of the subject is good. Therefore, the fourth figure D4, the fifth figure D5, and the sixth figure D6 are colored with light red. Also in FIG. 5, the number of hatched lines represents the shading of color. Thus, the display modes of the fourth figure D4, the fifth figure D5, and the sixth figure D6 are determined based on the physiological information and the reference information on the mean blood pressure.

By visually recognizing the image 200, the user can grasp that the mean blood pressure does not fluctuate greatly while the heart rate of the subject gradually increases. In such a case, since it is known that the mean blood pressure gradually decreases, the user can predict that the mean blood pressure of the subject gradually decreases in the future by visually recognizing the image 200.

Also in the present example, according to the information processing device 20, the same effects as or similar effects to those of the first example of the first embodiment are achieved.

According to the information processing device 20 having the above configuration, in the display data, the plurality of pieces of change tendency information generated for each piece of physiological information based on two types of physiological information are displayed side by side for each piece of physiological information. Therefore, for example, by visually recognizing the image 200 based on such display data, the user can grasp the change tendency related to the two types of physiological information, and thus can accurately grasp the condition of the subject.

### Third Example of First Embodiment

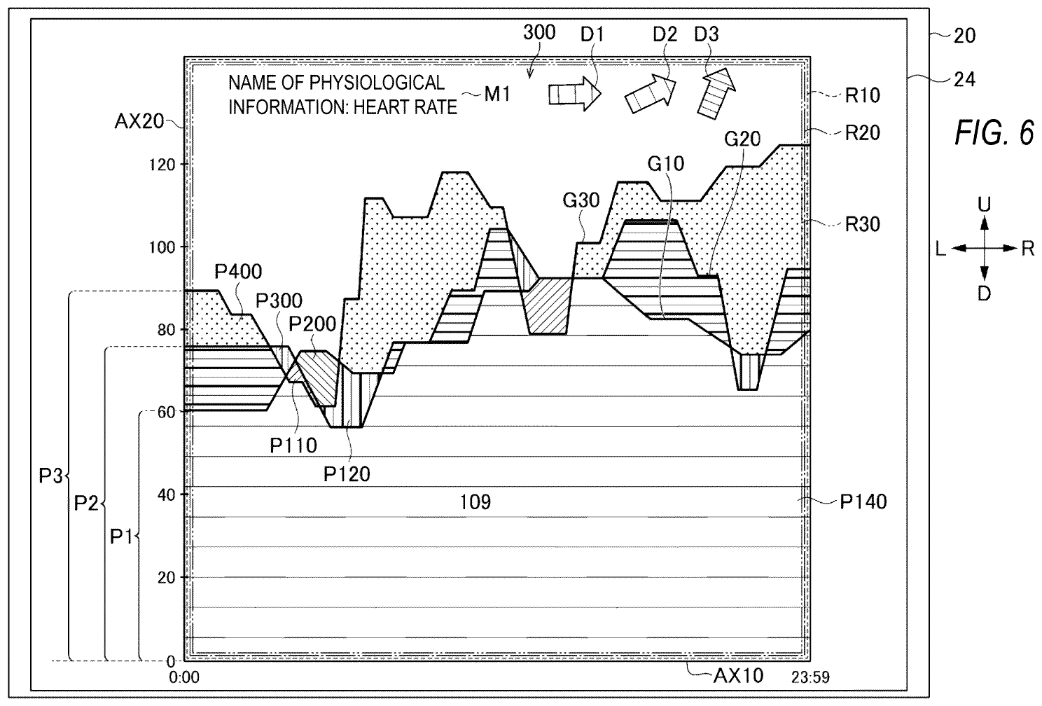

Next, a third example of the first embodiment will be described with reference to FIGS. 2 and 6. FIG. 6 illustrates an image 300 based on the display data. In the third example, parts the same or similar to those of the first example of the first embodiment are denoted by the same or similar reference numerals as those of the first example of the first embodiment, and description of repeated parts is omitted as appropriate. The present example is different from the first example of the first embodiment in that the display data is generated in a form in which the first physiological information group, the second physiological information group, and the third physiological information group are displayed with a first axis AX10 that is a timeline overlapping and a second axis AX20 that indicates the value related to the physiological information being common. The physiological information in the present example is the heart rate. Therefore, unless otherwise specified, in the present example, the “physiological information”, the “first physiological information group”, the “second physiological information group”, and the “third physiological information group” refer to heart rates.

In the present example, the display data can include a first graph object G10 (see FIG. 6) indicating the size of each piece of physiological information included in the first physiological information group, a second graph object G20 (see FIG. 6) indicating the size of each piece of physiological information included in the second physiological information group, and a third graph object G30 (see FIG. 6) indicating the size of each piece of physiological information included in the third physiological information group.

In the present example, the controller 23 is configured to specify a first portion P1 (see FIG. 6) surrounded by the first graph object G10 and the first axis AX10. The controller 23 is configured to specify a second portion P2 (see FIG. 6) surrounded by the second graph object G20 and the first axis AX10. The controller 23 is configured to specify a third portion P3 (see FIG. 6) surrounded by the third graph object G30 and the first axis AX10. The controller 23 is configured to specify an overlapped portion P110 (see FIG. 6) which is a portion where the first portion P1 and the second portion P2 overlap. The controller 23 is configured to specify an overlapped portion P120 (see FIG. 6) which is a portion where the first portion P1 and the third portion P3 overlap. The controller 23 is configured to specify an overlapped portion P130 (see FIG. 6) which is a portion where the second portion P2 and the third portion P3 overlap. The controller 23 is configured to specify an overlapped portion P140 (see FIG. 6) which is a portion where the first portion P1, the second portion P2, and the third portion P3 overlap. The controller 23 is configured to specify non-overlapped portions P200, P300, and P400 (see FIG. 6) which are portions where at least two of the first portion P1, the second portion P2, and the third portion P3 do not overlap (that is, portions other than the overlapped portions P110, P120, P130, and P140). The non-overlapped portion P200 is a part of the first portion P1, the non-overlapped portion P300 is a part of the second portion P2, and the non-overlapped portion P400 is a part of the third portion P3.

The controller 23 is configured to set colors to be colored in the overlapped portions P110, P120, P130, and P140 and the non-overlapped portions P200, P300, and P400 by adjusting RGB gradation. Note that RGB is so-called three primary colors of light, and R represents red, G represents green, and B represents blue. In the present example, the non-overlapped portion P200 is colored with a first color (blue), the non-overlapped portion P300 is colored with a second color (red), and the non-overlapped portion P400 is colored with a third color (green). The controller 23 is configured to set an average value of the gradation of each color of RGB in the first color and the gradation of each color of RGB in the second color as the gradation of each color of RGB in a fourth color to be colored in the overlapped portion P110. Therefore, the fourth color is a combination of blue and red (that is, purple). The controller 23 is configured to set an average value of the gradation of each color of RGB in the first color and the gradation of each color of RGB in the third color as the gradation of each color of RGB in a fifth color to be colored in the overlapped portion P120. Therefore, the fifth color is a combination of blue and green (that is, blue-green). The controller 23 is configured to set an average value of the gradation of each color of RGB in the second color and the gradation of each color of RGB in the third color as the gradation of each color of RGB in a sixth color to be colored in the overlapped portion P130. Therefore, the sixth color is a combination of red and green (that is, yellow). The controller 23 is configured to set an average value of the gradation of each color of RGB in the first color, the gradation of each color of RGB in the second color, and the gradation of each color of RGB in the third color as the gradation of each color of RGB in a seventh color to be colored in the overlapped portion P140. Therefore, the seventh color is a combination of blue, red, and green (that is, gray).

In the present example, the controller 23 is configured to executes the information processing method illustrated in FIG. 2. However, in the present example, the controller 23 is configured to generate graph object information based on the first physiological information group, graph object information based on the second physiological information group, and graph object information based on the third physiological information group. In addition, the controller 23 is configured to generate display data in a form in which the first physiological information group, the second physiological information group, and the third physiological information group are displayed with the first axis AX10 that is the timeline overlapping and the second axis AX20 that indicates the value related to the physiological information being common. Therefore, in the present example, the image 300 illustrated in FIG. 6 is displayed on the display unit 24.

As illustrated in FIG. 6, the image 300 can include the first name M1, the first axis AX10, the second axis AX20, the first graph object G10, the second graph object G20, the third graph object G30, the first figure D1, the second figure D2, and the third figure D3. The first axis AX10 is the timeline and extends in the lateral direction (the left-right direction in FIG. 6). The second axis AX20 extends in the vertical direction (the up-down direction in FIG. 6). The first graph object G10, the second graph object G20, and the third graph object G30 indicate the magnitudes of the heart rate. The first graph object G10 corresponds to the first physiological information group. The second graph object G20 corresponds to the second physiological information group. The third graph object G30 corresponds to the third physiological information group. In the present example, the first physiological information group, the second physiological information group, and the third physiological information group are displayed in a state where a first displayable region R10 in which the first physiological information group is displayable, a second displayable region R20 in which the second physiological information group is displayable, and a third displayable region R30 in which the third physiological information group is displayable overlap each other on the same timeline (that is, on the first axis AX10).

In the present example, the non-overlapped portion P200 is colored with blue, the non-overlapped portion P300 is colored with red, and the non-overlapped portion P400 is colored with green. The overlapped portion P110 is colored purple, the overlapped portion P120 is colored blue-green, the overlapped portion P130 is colored yellow, and the overlapped portion P140 is colored gray. Note that, in FIG. 6, a hatched portion with diagonal lines downward to the right indicates a portion colored with blue. A hatched portion of vertical lines indicates a portion colored with red. A dotted hatched portion indicates a portion colored with green. A hatched portion with diagonal lines upward to the right indicates a portion colored with purple. A hatched portion with vertical lines including a thin line and a thick line indicates a portion colored with blue-green. A hatched portion with horizontal lines including a thin line and a thick line indicates a portion colored with yellow. A hatched portion with a horizontal line indicates a portion colored with gray. As described above, the display modes of the overlapped portions P110, P120, P130, and P140 are different from the display modes of the non-overlapped portions P200, P300, and P400 (portions including only the first portion P1, the second portion P2, and the third portion P3). Accordingly, the user can easily distinguish the overlapped portions P110, P120, P130, and P140 from the non-overlapped portions P200, P300, and P400. In addition, regarding the overlapped portions P110, P120, P130, and P140, two or more portions that form the overlapping among the first portion P1, the second portion P2, and the third portion P3 are each colored in a color associated with each of them. Therefore, the user can intuitively recognize that the overlapped portions P110, P120, P130, and P140 are portions where at least two of the first portion P1, the second portion P2, and the third portion P3 overlap.

An average value of values indicated by the physiological information (in the present embodiment, the heart rate) included in the third physiological information group is displayed in a lower center of the image 300. Therefore, in the present example, a numeral “109” indicating the average value is displayed in the lower center of the image 300. In the image 300, a location where the average value of the values indicated by the physiological information included in the third physiological information group is displayed is not limited to the lower center of the image 300. The value displayed in the lower center of the image 300 may be, for example, a value indicated by physiological information at any time point during the third period.

Also in the present example, according to the information processing device 20, the same effects as or similar effects to those of the first example of the first embodiment are achieved.

According to the information processing device 20 having the above configuration, the display data includes the first axis AX10 that is the timeline, and the second axis AX20 that indicates the magnitude of the value of the physiological information on the subject. The display data is generated in a form in which the first physiological information group, the second physiological information group, and the third physiological information group are displayed with the first axis AX10 overlapping and the second axis AX20 being common. Therefore, for example, the user can easily notice the difference between the first physiological information group and the second physiological information group and the difference between the second physiological information group and the third physiological information group by visually recognizing the image 300 based on such display data.

According to the information processing device 20 having the above configuration, the first graph object G10, the second graph object G20, the third graph object G30, the first figure D1, the second figure D2, and the third figure D3 are displayed in the same screen (in the image 300). Therefore, by visually recognizing the image 300, the user can quickly recognize the change tendency of the physiological information on the subject during each period, the difference between the periods, and the change tendency of the physiological information on the subject when comparing the previous and subsequent periods.

### Fourth Example of First Embodiment

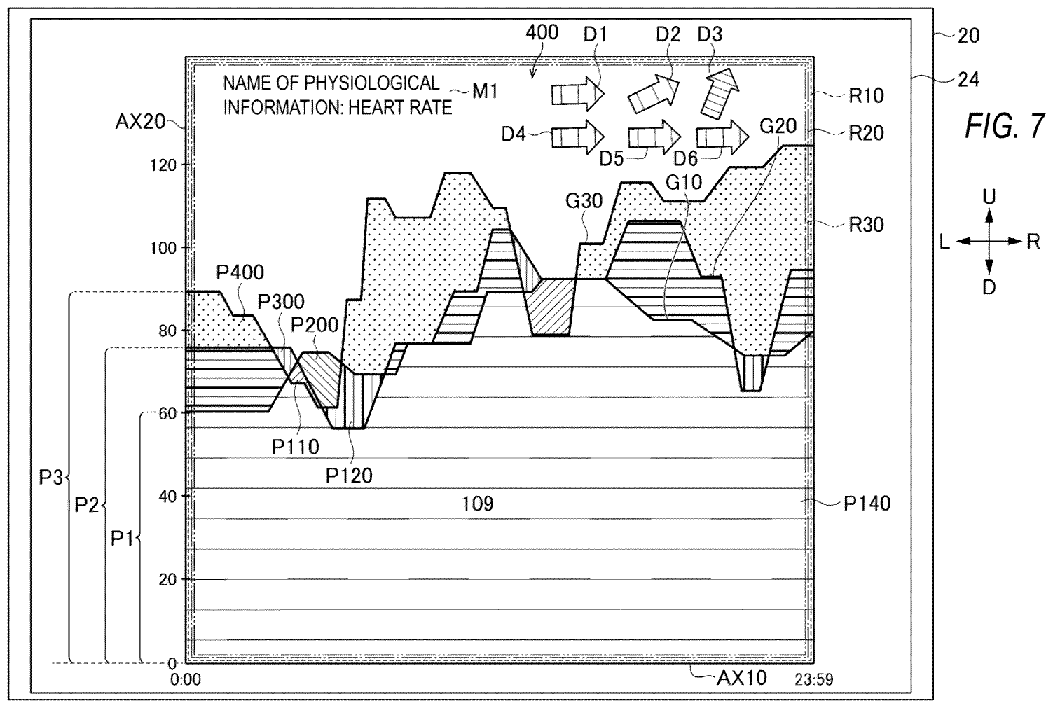

Next, a fourth example of the first embodiment will be described with reference to FIGS. 4 and 7. FIG. 7 illustrates an image 400 based on the display data. In the present example, the same reference numerals as those in the first example to the third example of the first embodiment will be used for the same parts as those in the first example to the third example of the first embodiment, and the description of the overlapping parts will be omitted as appropriate. The present example is different from the second example of the first embodiment in that the display data is generated in a form in which the first physiological information group, the second physiological information group, and the third physiological information group on the heart rate are displayed with the first axis AX10 that is the timeline overlapping and the second axis AX20 that indicates the value related to the physiological information being common. In the present example, the controller 23 also executes information processing same as or similar to the information processing in the third example of the first embodiment.

In the present example, the controller 23 is configured to execute the information processing method illustrated in FIG. 4. However, in the present example, the controller 23 is configured to generates display data including the first physiological information group related to the heart rate, the second physiological information group related to the heart rate, and the third physiological information group related to the heart rate. The controller 23 is configured to generate the display data in a form in which the first physiological information group, the second physiological information group, and the third physiological information group related to the heart rate are displayed with the first axis AX10 overlapping and the second axis AX20 being common. Therefore, in the present example, the image 400 illustrated in FIG. 7 is displayed on the display unit 24.

As illustrated in FIG. 7, the image 400 is different from the image 300 in that the image 400 includes arrow-shaped figures (the fourth figure D4, the fifth figure D5, and the sixth figure D6) indicating a change tendency related to the mean blood pressure. The fourth figure D4 is displayed directly below the first figure D1. The fifth figure D5 is displayed directly below the second figure D2. The sixth figure D6 is displayed directly below the third figure D3.

Also in the present example, according to the information processing device 20, the same effects as or similar effects to those of the first example to the third example of the first embodiment are achieved.

According to the information processing device 20 having the above configuration, the image 400 can include the first graph object G10, the second graph object G20, the third graph object G30, the first figure D1, the second figure D2, the third figure D3, the fourth figure D4, the fifth figure D5, and the sixth figure D6. That is, the image 400 also includes arrow-shaped figures indicating a change tendency of the physiological information (the mean blood pressure) different from the physiological information (the heart rate) indicated by the first graph object G10, the second graph object G20, and the third graph object G30. As described above, by displaying the graph object related to the mean blood pressure together with the graph object related to the heart rate and the arrow-shaped figure on the display unit 24, it is possible to prevent the number of displayed graph objects from increasing and the visibility from decreasing, and the user can accurately grasp the condition of the subject.

### Second Embodiment

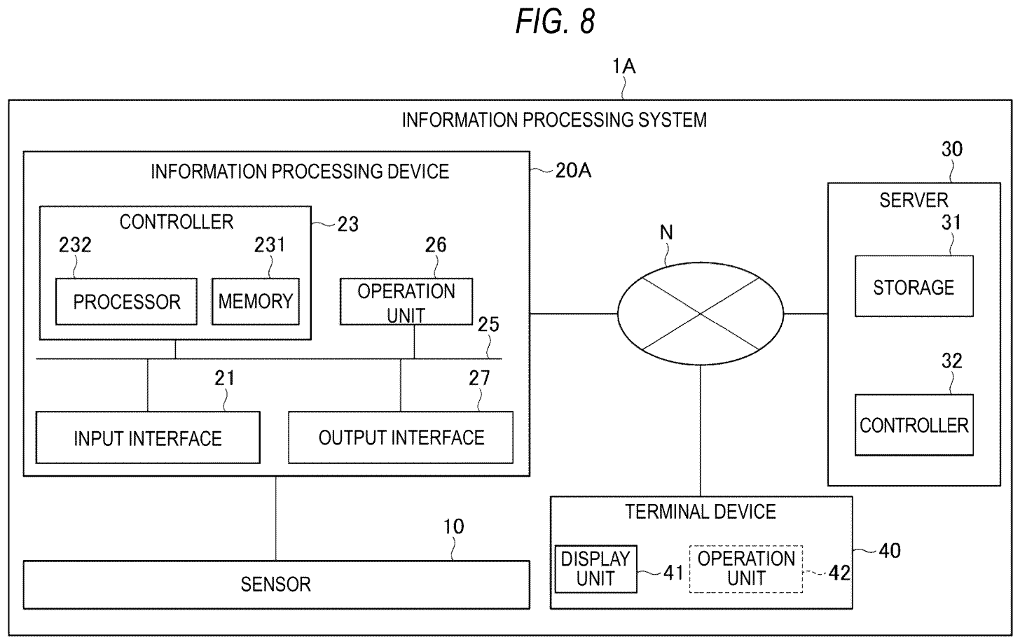

Next, an information processing system 1A according to a second embodiment will be described with reference to FIG. 8. In the present embodiment, portions similar to those of the first embodiment will be described using the same reference numerals as those of the first embodiment, and description of overlapped portions will be appropriately omitted. The present embodiment is different from the first embodiment in that the information processing system 1A includes an information processing device 20A instead of the information processing device 20 and also includes a server 30 and a terminal device 40.

FIG. 8 is a schematic diagram of the information processing system 1A according to the second embodiment. The information processing system 1A is used in, for example, a medical facility such as a hospital. As illustrated in FIG. 8, the information processing system 1A can include the sensor 10, the information processing device 20A, the server 30, and the terminal device 40. The sensor 10 and the information processing device 20A are connected by wire or wirelessly. The information processing device 20A, the server 30, and the terminal device 40 are communicably connected via the network N. The network Nis, for example, a computer network such as a cloud network. The cloud network is a computer network existing in an infrastructure of cloud computing.

The information processing device 20A can include the input interface 21, the controller 23, an operation unit 26, and an output interface 27. These components are communicably connected to each other via the bus 25. In the present embodiment, the controller 23 is configured to transmit the display data and the like to the output interface 27.

The operation unit 26 is configured to receive an input operation from a user such as a medical worker. The operation unit 26 is, for example, a touch panel disposed on the display unit 24 in an overlapping manner, an operation button attached to a housing, or the like. For example, in a case where the user performs an input operation, on the operation unit 26, for obtaining desired information from the server 30, the operation unit 26 generates a request signal for obtaining desired information from the server 30, based on the input operation. The operation unit 26 is configured to transmit the generated request signal to the server 30.

The output interface 27 is configured to output, to the terminal device 40, the display data and the like received from the controller 23. For example, the output interface 27 is configured to output, to the terminal device 40, an output signal corresponding to display data and the like. The output interface 27 may include a circuit configured to convert output data into an output signal that can be processed by the terminal device 40, as necessary.

The server 30 is, for example, a server computer on a cloud network. The server 30 can include a storage 31 and a controller 32.

The storage 31 may have the same hardware configuration as the storage 22 of the information processing device 20. The storage 31 stores, for example, various types of information same as or similar to the various types of information stored in the storage 22 in the first embodiment.

The controller 32 may have the same hardware configuration as the controller 23. For example, the controller 32 transmits all or some of the various types of information stored in the storage 31 to the information processing device 20A based on the request signal transmitted from the operation unit 26 of the information processing device 20A.

The terminal device 40 is, for example, an electronic device such as a desktop type PC, a notebook PC, a tablet terminal, or a smartphone. The terminal device 40 can include a display unit 41.

The display unit 41 is, for example, a touch screen display such as a liquid crystal display or an organic EL display. The display unit 41 is configured to display, for example, the display data and the like received from the information processing device 20A.

In the present embodiment, the information processing device 20A executes the information processing method illustrated in FIG. 2 or FIG. 4. However, the controller 23 of the information processing device 20A is configured to transmit the generated display data to the output interface 27. The output interface 27 is configured to output the display data received from the controller 23 to the terminal device 40. Therefore, in the present embodiment, the images 100, 200, 300, 400 are displayed on the display unit 41 of the terminal device 40.

The information processing device 20A according to the above configuration also achieves the same effects as or similar effects to the information processing device 20 according to the first embodiment.

The foregoing description of the exemplary embodiments of the present invention has been provided for the purposes of illustration and description. It is not intended to be exhaustive or to limit the invention to the precise forms disclosed. Obviously, many modifications and variations will be apparent to practitioners skilled in the art. The embodiments were chosen and described in order to best explain the principles of the invention and its practical applications, thereby enabling others skilled in the art to understand the invention for various embodiments and with the various modifications as are suited to the particular use contemplated. It is intended that the scope of the invention be defined by the following claims and their equivalents.

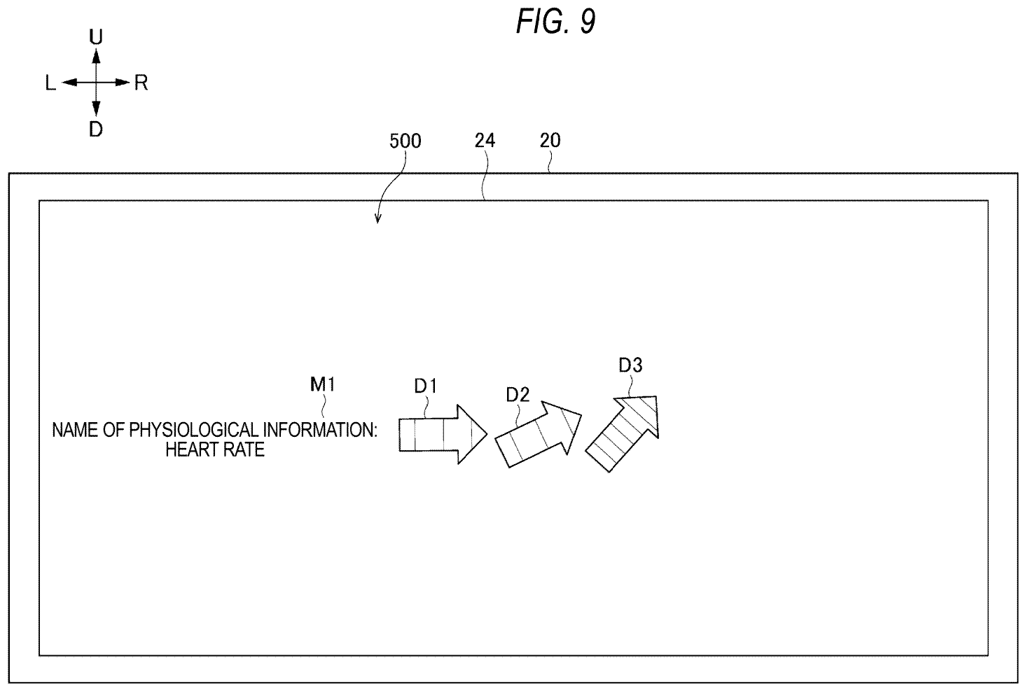

In the above embodiments, the display data includes the physiological information (including the case of the first physiological information group, the second physiological information group, and the third physiological information group) and the name information, but the display data may not include at least one of the physiological information and the name information. For example, in the first example of the first embodiment, when the physiological information is not included in the display data, an image 500 illustrated in FIG. 9 is displayed on the display unit 24 of the information processing device 20.

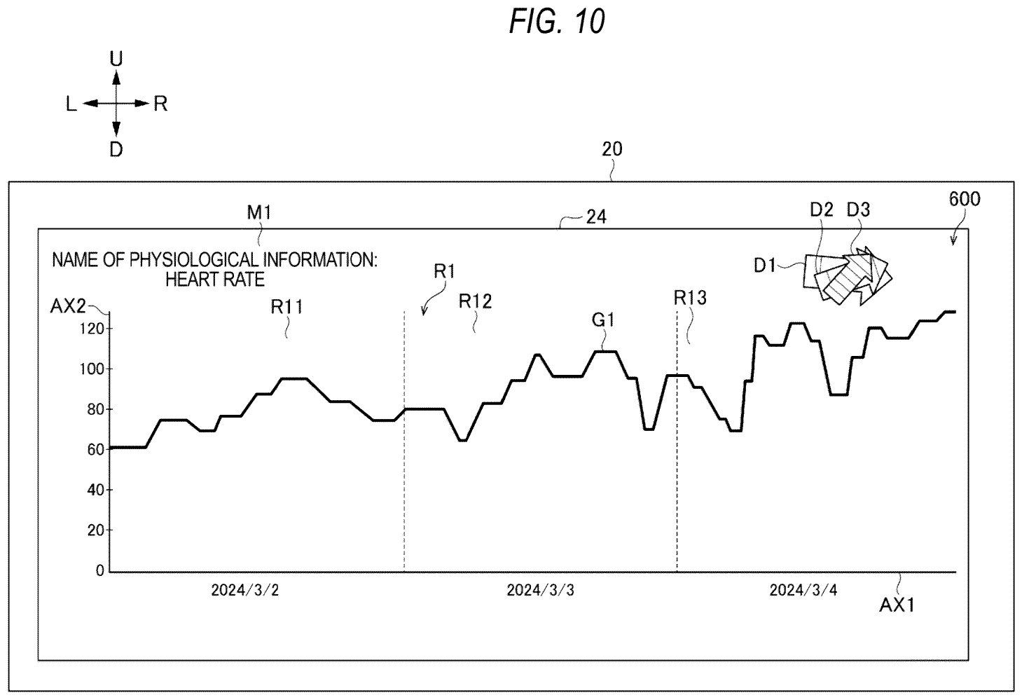

In the above embodiments, the controller 23 generates display data in which a plurality of pieces of change tendency information are displayed side by side, but may generate, for example, display data in which the plurality of pieces of change tendency information are displayed in an overlapping manner. For example, in the first example of the first embodiment, when the controller 23 generates the display data in which the plurality of pieces of change tendency information are displayed in an overlapping manner, an image 600 illustrated in FIG. 10 is displayed on the display unit 24 of the information processing device 20. In this case, as illustrated in FIG. 10, the sizes of the arrow-shaped figures may be different.

In the above embodiments, the first value is the total value of the values indicated by the pieces of physiological information included in the first physiological information group, the second value is the total value of the values indicated by the pieces of physiological information included in the second physiological information group, and the third value is the total value of the values indicated by the pieces of physiological information included in the third physiological information group, but the first value, the second value, and the third value are not limited to this example. For example, the first value may be an average value of values obtained by performing moving average processing on the first physiological information group, the second value may be an average value of values obtained by performing moving average processing on the second physiological information group, and the third value may be an average value of values obtained by performing moving average processing on the third physiological information group. In this case, according to the information processing devices 20, 20A, the first change tendency information and the second change tendency information can be generated by relatively simple information processing.

In the above embodiments, the controller 23 is configured to classify the physiological information input to the input interface 21 into the first physiological information group, the second physiological information group, and the third physiological information group, but the present disclosure is not limited thereto. For example, the controller 23 may be configured to classify the physiological information input to the input interface 21 into the first physiological information group, the second physiological information group, the third physiological information group, and a fourth physiological information group. That is, the controller 23 may be configured to classify the physiological information input to the input interface 21 into four or more physiological information groups.

In the second embodiment, the information processing system 1A includes the sensor 10, the information processing device 20A, the server 30, and the terminal device 40, but may include a central monitor instead of the server 30. In this case, the central monitor has the same function as the server 30.

In the third example and the fourth example of the first embodiment, the overlapped portions P110, P120, P130, and P140 and the non-overlapped portions P200, P300, and P400 are colored with different colors, but the overlapped portions P110, P120, P130, and P140 and the non-overlapped portions P200, P300, and P400 may not be colored. In this case, the display modes of the first graph object G10, the second graph object G20, and the third graph object G30 may be different. For example, the first graph object G10 may be a graph having a solid line, the second graph object G20 may be a graph having a broken line, and the third graph object G30 may be a graph having a one-dot chain line.

In the above embodiments, the initial change tendency information is generated based on the value obtained by subtracting the value indicated by the physiological information input to the input interface 21 at the input start time from the value indicated by the physiological information input to the input interface 21 after the predetermined time (one hour in the above embodiments) from the input start time, but the present disclosure is not limited thereto. For example, the initial change tendency information may be generated based on a value obtained by dividing a value indicated by physiological information input to the input interface 21 after a predetermined time from the input start time by a value indicated by physiological information input to the input interface 21 at the input start time.

In the above embodiments, the first comparison value is the difference between the first value and the second value (the value obtained by subtracting the first value from the second value), and the second comparison value is the difference between the second value and the third value (the value obtained by subtracting the second value from the third value), but the present disclosure is not limited thereto. For example, the first comparison value may be a value obtained by dividing the second value by the first value, and the second comparison value may be a value obtained by dividing the third value by the second value. For example, the first comparison value may be a value obtained by subtracting an average value (a first average value) of values indicated by the pieces of physiological information included in the first physiological information group from an average value (a second average value) of values indicated by the pieces of physiological information included in the second physiological information group, and the second comparison value may be a value obtained by subtracting the second average value from an average value (a third average value) of values indicated by the pieces of physiological information included in the third physiological information group. For example, the first comparison value may be a value obtained by dividing the second average value by the first average value, and the second comparison value may be a value obtained by dividing the third average value by the second average value. For example, the first comparison value may be a value obtained by subtracting a median (a first median) of the values indicated by the pieces of physiological information included in the first physiological information group from a median (a second median) of the values indicated by the pieces of physiological information included in the second physiological information group, and the second comparison value may be a value obtained by subtracting the second median from a median (a third median) of the values indicated by the pieces of physiological information included in the third physiological information group. For example, the first comparison value may be a value obtained by dividing the second median value by the first median value, and the second comparison value may be a value obtained by dividing the third median value by the second median value. For example, the first comparison value may be the largest difference (maximum difference) among the differences between the values indicated by the physiological information at each time in the first physiological information group and the second physiological information group, and the second comparison value may be the largest difference (maximum difference) among the differences between the values indicated by the physiological information at each time in the second physiological information group and the third physiological information group.

In the above embodiments, the physiological information during the period from 0:00 on Mar. 2, 2024 to 23:59 on Mar. 4, 2024 is input to the input interface 21, but the present disclosure is not limited thereto. For example, the physiological information during a period from 0:00 on Mar. 3, 2024 to 23:59 on Mar. 5, 2024 may be input to the input interface 21. In this case, the controller 23 is configured to classify the physiological information during the period from 0:00 on Mar. 3, 2024 to 23:59 on Mar. 5, 2024 into the first physiological information group including the physiological information on Mar. 3, 2024, the second physiological information group including the physiological information on Mar. 4, 2024, and the third physiological information group including the physiological information on Mar. 5, 2024. In this case, the controller 23 generates a plurality of pieces of change tendency information for the most recent three days from a day to which the current time belongs.

In the above embodiments, the controller 23 generates the condition information based on whether the number of pieces of physiological information indicating values outside the normal range in a day is equal to or greater than the predetermined value, but the present disclosure is not limited thereto. For example, the controller 23 may generate the condition information based on whether the first value, the second value, or the third value is equal to or greater than a predetermined value.

In the above embodiments, when the condition information indicates that the condition of the subject is good, the arrow-shaped figure corresponding to the change tendency information during the period corresponding to the condition information is colored with light red, whereas when the condition information indicates that the condition of the subject is poor, the arrow-shaped figure corresponding to the change tendency information during the period corresponding to the condition information is colored with dark red. For example, when the condition information indicates that the condition of the subject is good, the arrow-shaped figure corresponding to the change tendency information during the period corresponding to the condition information may be colored with blue, whereas when the condition information indicates that the condition of the subject is poor, the arrow-shaped figure corresponding to the change tendency information during the period corresponding to the condition information may be colored with red. For example, when the condition information indicates that the condition of the subject is good, the arrow-shaped figure corresponding to the change tendency information during the period corresponding to the condition information may be displayed in a first pattern, whereas when the condition information indicates that the condition of the subject is poor, the arrow-shaped figure corresponding to the change tendency information during the period corresponding to the condition information may be displayed in a second pattern.

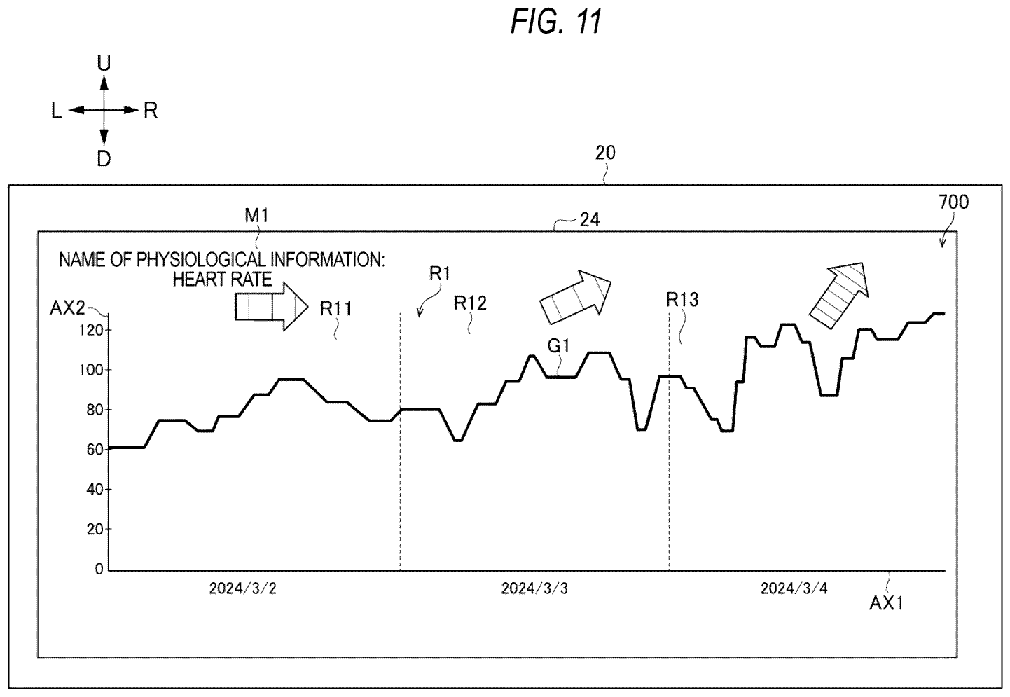

In the above embodiments, the display positions of the first figure D1, the second figure D2, the third figure D3, the fourth figure D4, the fifth figure D5, and the sixth figure D6 are not limited to the positions described in the above embodiments. For example, the first figure D1, the second figure D2, the third figure D3, the fourth figure D4, the fifth figure D5, and the sixth figure D6 may be displayed directly above the heart rate graph object G1, the mean blood pressure graph object G2, the first graph object G10, the second graph object G20, and the third graph object G30. For example, when such display is performed in the first example of the first embodiment, an image 700 illustrated in FIG. 11 is displayed on the display unit 24 of the information processing device 20. As illustrated in FIG. 11, in the image 700, the first figure D1 is displayed directly above the graph object corresponding to the physiological information on the heart rate on Mar. 2, 2024, of the heart rate graph object G1. The second figure D2 is displayed directly above the graph object corresponding to the physiological information on the heart rate on Mar. 3, 2024, of the heart rate graph object G1. The third figure D3 is displayed directly above the graph object corresponding to the physiological information on the heart rate on Mar. 4, 2024, of the heart rate graph object G1. That is, in this case, the first figure D1 is displayed in the left region R11, the second figure D2 is displayed in the central region R12, and the third figure D3 is displayed in the right region R13.

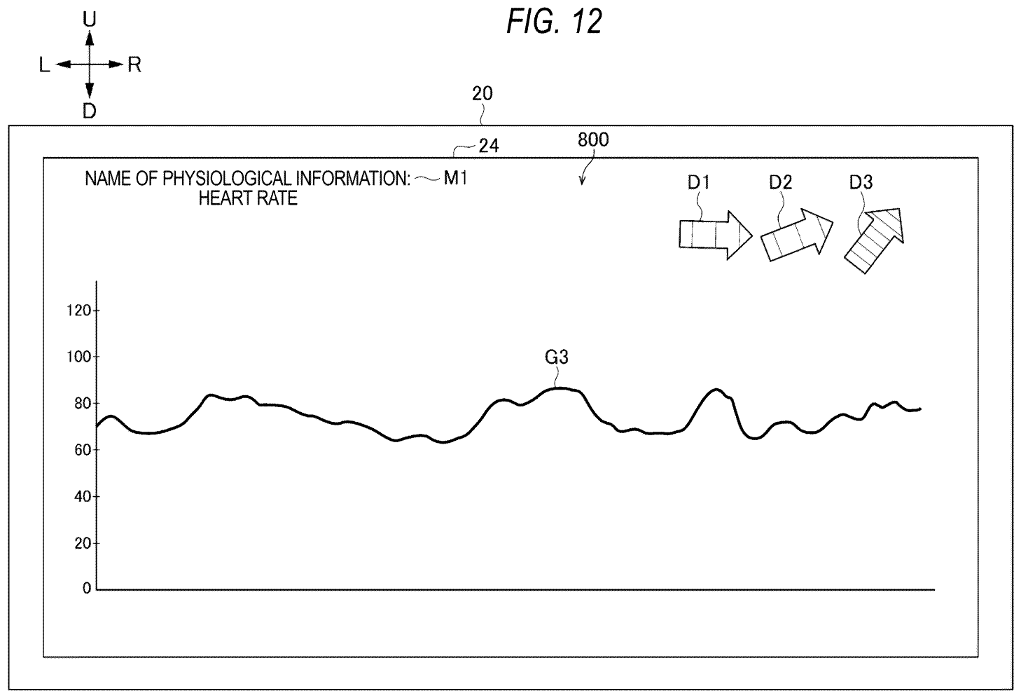

In the above embodiments, the display data includes the physiological information (including the case of the first physiological information group, the second physiological information group, and the third physiological information group), and the images 100, 200, 300, 400 include the graph object corresponding to the physiological information, but the present disclosure is not limited thereto. For example, the display data may include physiological waveform information instead of physiological information, and the images 100, 200, 300, 400 may include a physiological waveform graph object corresponding to the physiological waveform information. In this case, the controller 23 generates the physiological waveform information based on the physiological information input to the input interface 21. Then, the controller 23 generates the display data including the physiological waveform information. For example, in the first example of the first embodiment, when the display data includes the physiological waveform information instead of the physiological information, an image 800 illustrated in FIG. 12 is displayed on the display unit 24 of the information processing device 20. As illustrated in FIG. 12, the image 800 includes, for example, a physiological waveform graph object G3 indicating physiological waveform information in one minute immediately before the current time. The first figure D1, the second figure D2, and the third figure D3 are displayed above the physiological waveform graph object G3.

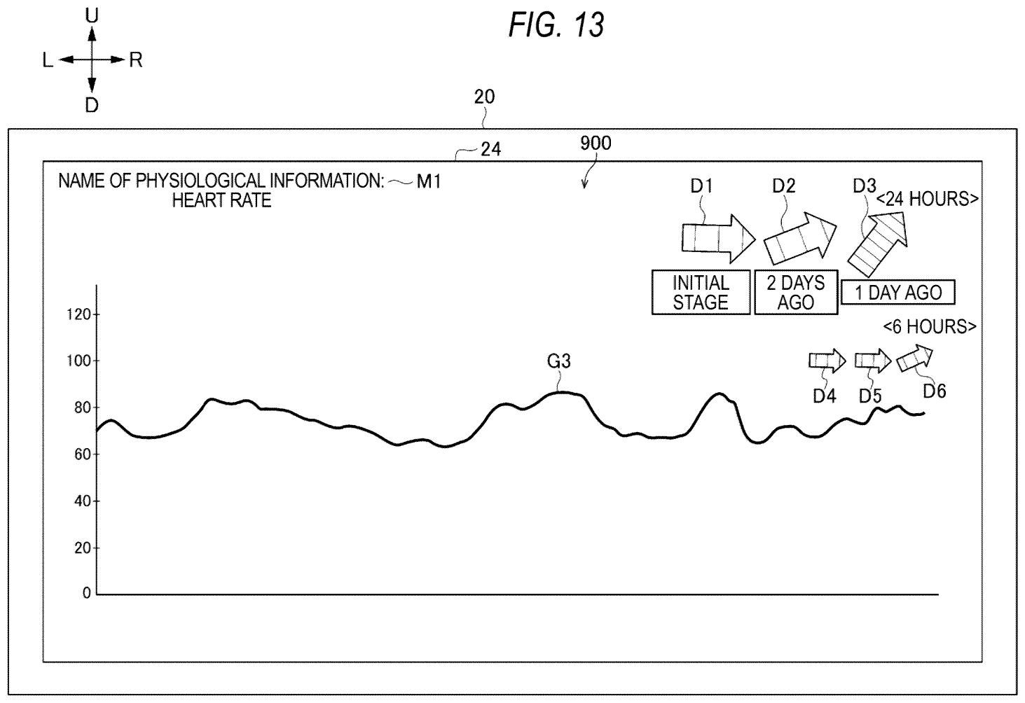

In the above embodiments, as illustrated in FIG. 13, an image 900 may be displayed on the display unit 24 of the information processing device 20. In the image 900, the fourth figure D4, the fifth figure D5, and the sixth figure D6 are displayed as the change tendency information in addition to the first figure D1, the second figure D2, and the third figure D3 included in the image 800 illustrated in FIG. 12. The fourth figure D4, the fifth figure D5, and the sixth figure D6 are figures each having an inclination, and indicate a change tendency of the physiological information from one period to the other period by the inclination.

For example, as described above, 24 hours immediately before the current time is set as the third period, 24 hours immediately before the third period is set as the second period, and 24 hours immediately before the second period is set as the first period. 6 hours immediately before the current time are defined as a sixth period, 6 hours immediately before the sixth period are defined as a fifth period, and 6 hours immediately before the fifth period are defined as a fourth period.

In this case, in the image 900, for example, similarly to the image 100 illustrated in FIG. 3, the initial change tendency information is displayed by the first figure D1. Further, for example, the first change tendency information based on the difference between the first value obtained by performing the moving average processing on the first physiological information group obtained during the first period and the second value obtained by performing the moving average processing on the second physiological information group obtained during the second period is displayed by the second figure D2. Further, for example, the second change tendency information based on the difference between the second value and the third value obtained by performing the moving average processing on the third physiological information group obtained during the third period is displayed by the third figure D3.

For example, third change tendency information based on the difference between the third value and a fourth value obtained by performing the moving average processing on the fourth physiological information group obtained during the fourth period is displayed by the fourth figure D4. Further, for example, fourth change tendency information based on the difference between the fourth value and a fifth value obtained by performing the moving average processing on a fifth physiological information group obtained during the fifth period is displayed by the fifth figure D5. Further, for example, fifth change tendency information based on the difference between the fifth value and a sixth value obtained by performing the moving average processing on a sixth physiological information group obtained during the sixth period is displayed by the sixth figure D6.

In the example illustrated in FIG. 13, the lengths of the first period, the second period, and the third period are 24 hours, whereas the lengths of the fourth period, the fifth period, and the sixth period are 6 hours. As described above, the lengths of the plurality of periods corresponding to the plurality of pieces of change tendency information may be different from each other. As described above, the physiological waveform graph object G3 indicates physiological waveform information in one minute immediately before the current time. In this way, the length of the period corresponding to the change tendency information (24 hours or 6 hours in the example illustrated in FIG. 13) and the length of the period corresponding to the graph object displayed in the image 900 (one minute in the example illustrated in FIG. 13) may be different.

In the example illustrated in FIG. 13, the 24 hours (the third period) immediately before the current time includes the 6 hours (the sixth period) immediately before the current time, the 6 hours (the fifth period) immediately before the sixth period, and the 6 hours (the fourth period) immediately before the fifth period. As described above, at least a part of the plurality of periods respectively corresponding to the plurality of pieces of change tendency information may overlap.

The period corresponding to each piece of change tendency information may be displayed in a recognizable manner. For example, in the example illustrated in FIG. 13, characters “initial stage” indicating the initial change tendency information are displayed near the first figure D1. In addition, characters “24 hours” indicating that the length of the corresponding period is 24 hours are displayed near the second figure D2 and the third figure D3. In addition, characters “2 days ago” indicating that the corresponding period (the second period) is two days ago is displayed near the second figure D2. In addition, characters “1 day ago” indicating that the corresponding period (the third period) is one day ago is displayed near the third figure D3.

Characters “6 hours” indicating that the length of the corresponding period is 6 hours are displayed near the fourth figure D4, the fifth figure D5, and the sixth figure D6. Similarly, the change tendency information such as the first figure D1, the second figure D2, and the third figure D3 illustrated in FIGS. 3, 5, 6, and the like may be displayed such that the corresponding period is recognizable.

In the image 900 illustrated in FIG. 13, as an example, three figures (the fourth figure D4, the fifth figure D5, and the sixth figure D6) in which the length of the target period is 6 hours are displayed, but the number of these figures is not limited to three.

In the image 900, as an example, the fourth figure D4, the fifth figure D5, and the sixth figure D6 smaller than the third figure D3 are displayed side by side below the third figure D3, but the present disclosure is not limited to such a configuration. For example, each of the fourth figure D4, the fifth figure D5, and the sixth figure D6 may have the same size as the third figure D3.

The fourth figure D4, the fifth figure D5, and the sixth figure D6 may be displayed side by side in the lateral direction of the third figure D3. Further, a part of the fourth figure D4, the fifth figure D5, and the sixth figure D6, for example, only the sixth figure D6 may be displayed side by side in the lateral direction of the third figure D3.

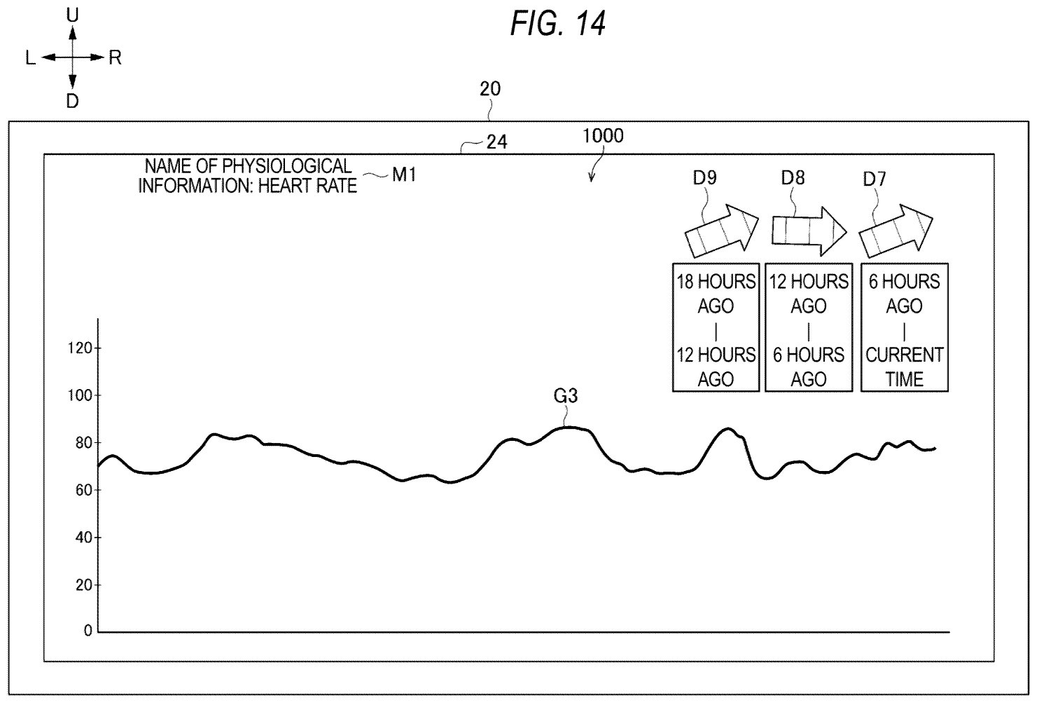

In the above embodiments, as illustrated in FIG. 14, an image 1000 may be displayed on the display unit 24 of the information processing device 20. In the image 800 illustrated in FIG. 12 described above, the change tendency information indicating the change tendency of the physiological information from one period to the other period is displayed. On the other hand, in the image 1000 illustrated in FIG. 14, the change tendency information indicating the change tendency of the physiological information during the one period is displayed.

Specifically, in the image 1000, a seventh figure D7, an eighth figure D8, and a ninth figure D9 are displayed as the change tendency information. The seventh figure D7, the eighth figure D8, and the ninth figure D9 are figures each having an inclination, and indicate a change tendency of the physiological information during the one period by the inclination.

As described above, 6 hours immediately before the current time are set as the sixth period, 6 hours immediately before the sixth period are set as the fifth period, and 6 hours immediately before the fifth period are set as the fourth period. In this case, in the image 1000, for example, when the value obtained by subtracting the value indicated by the physiological information at an end time of the fourth period from the value indicated by the physiological information at a start time of the fourth period is equal to or greater than +10, the seventh figure D7 indicating the change tendency of the physiological information during the fourth period is inclined upward and displayed according to the value. Further, for example, when the subtracted value is −10 or more and less than +10, the seventh figure D7 is displayed with zero inclination according to the value. Further, for example, when the subtracted value is less than −10, the seventh figure D7 is inclined downward and displayed according to the value.

Similarly, for the fifth period, for example, the inclination of the eighth figure D8 indicating the change tendency of the physiological information during the fifth period is determined according to a value obtained by subtracting the value indicated by the physiological information at an end time of the fifth period from the value indicated by the physiological information at a start time of the fifth period. Similarly, for the sixth period, for example, the inclination of the ninth figure D9 indicating the change tendency of the physiological information during the sixth period is determined according to a value obtained by subtracting the value indicated by the physiological information at an end time of the sixth period from the value indicated by the physiological information at a start time of the sixth period.

In the example illustrated in FIG. 14, the seventh figure D7 is inclined upward, and indicates that the change tendency of the physiological information during the sixth period is upward. The eighth figure D8 has the inclination of zero and indicates that the change tendency of the physiological information during the fifth period is flat. The ninth figure D9 is inclined upward and indicates that the change tendency of the physiological information during the fourth period is upward.

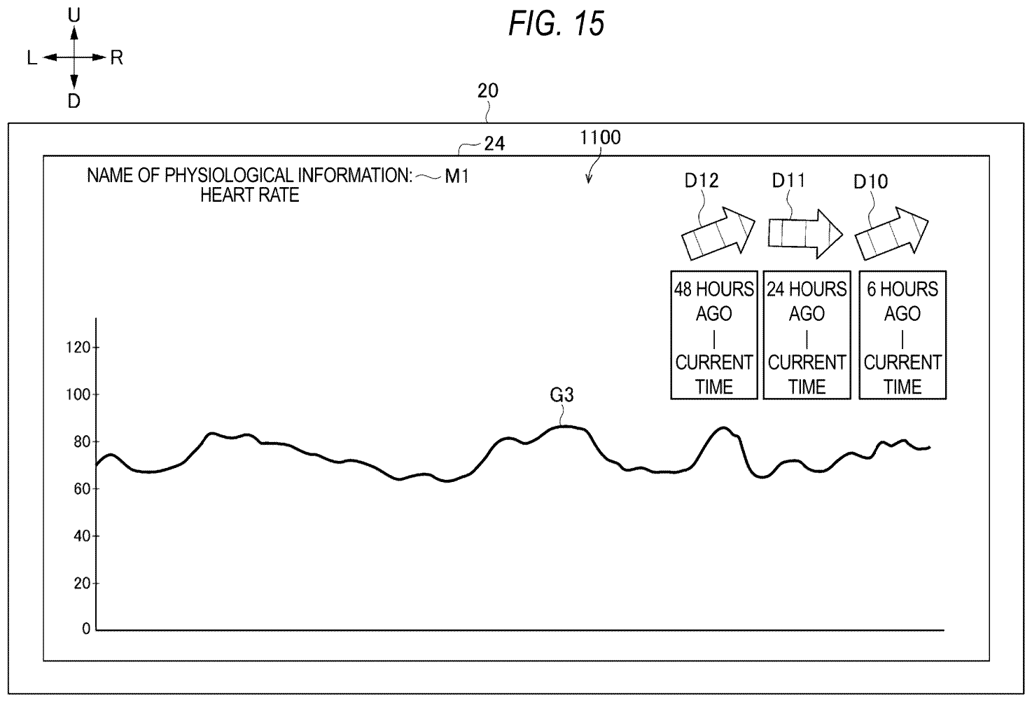

In the above embodiments, as illustrated in FIG. 15, an image 1100 may be displayed on the display unit 24 of the information processing device 20. In the image 1100, a tenth figure D10, an eleventh figure D11, and a twelfth figure D12 are displayed as the change tendency information. The tenth figure D10, the eleventh figure D11, and the twelfth figure D12 are figures each having an inclination, and indicate a change tendency of the physiological information during the one period by the inclination.

The seventh figure D7, the eighth figure D8, and the ninth figure D9 illustrated in FIG. 14 described above have different periods indicating the change tendency of the physiological information. In contrast, in the tenth figure D10, the eleventh figure D11, and the twelfth figure D12 illustrated in FIG. 15, the periods indicating the change tendency of the physiological information overlap each other.

In the example illustrated in FIG. 15, the tenth figure D10 indicates a change tendency of the physiological information in 6 hours immediately before the current time. In addition, the eleventh figure D11 indicates a change tendency of the physiological information in 24 hours immediately before the current time. In addition, the twelfth figure D12 indicates a change tendency of the physiological information in 48 hours immediately before the current time.

As illustrated in FIGS. 12 and 13, in a case where the display data includes the physiological waveform information, the user can grasp the physiological waveform information during a short period such as the latest one minute, for example, in addition to the change tendency of the physiological information on the subject over a long period. In addition, the user can grasp the change tendency of the physiological information for each period by visually recognizing the images 1000, 1100 as illustrated in FIGS. 14 and 15. Therefore, for example, the user can more accurately grasp the change tendency of the physiological information on the subject by visually recognizing the images 800, 900, 1000, 1100 based on such display data.

In the second example and the fourth example of the first embodiment, two types of physiological information (the heart rate and the mean blood pressure) are input to the input interface 21, but three or more types of physiological information may be input.

In the third example and the fourth example of the first embodiment, the controller 23 sets the colors to be colored in the overlapped portions P110, P120, P130, and P140 and the non-overlapped portions P200, P300, and P400 by adjusting the RGB gradation, but the present disclosure is not limited thereto. For example, the controller 23 may use an xy chromaticity diagram to set colors to be colored in the overlapped portions P110, P120, P130, and P140 and the non-overlapped portions P200, P300, and P400.

In the fourth example of the first embodiment, the image 400 may include not only the first name M1 but also the second name M2. The image 400 may include not only the first graph object G10, the second graph object G20, and the third graph object G30 but also a graph object related to the mean blood pressure.

In the second embodiment, the controller 32 of the server 30 transmits all or some of the various types of information stored in the storage 31 to the information processing device 20A based on the request signal transmitted from the operation unit 26 of the information processing device 20A, but the present disclosure is not limited thereto. For example, the controller 32 may transmit all or some of the various types of information stored in the storage 31 to the information processing device 20A based on a request signal transmitted from an operation unit 42 (see a broken line portion in FIG. 8) included in the terminal device 40. In this case, the operation unit 42 is, for example, a touch panel disposed on the display unit 41 in an overlapping manner, an operation button attached to a housing, or the like, and is configured to receive an input operation by the user.

As described above, the present specification discloses the following matters.

(1) An information processing device including:
at least one processor; and
one or more memories configured to store at least one instruction to be executed by the at least one processor,
wherein when the at least one instruction is executed by the processor, the information processing device is configured to:
   generate, based on physiological information on a subject, a plurality of pieces of change tendency information indicating a change tendency of the physiological information during one period or a change tendency of the physiological information from one period to an other period; and
   generate display data in which the plurality of pieces of change tendency information are displayed side by side or the plurality of pieces of change tendency information are displayed so as to be overlapped with each other.

(2) The information processing device according to (1),
in which in the display data, the change tendency information is displayed as a figure having an inclination,
the inclination indicates an amount of change of the physiological information during the one period or an amount of change of the physiological information from the one period to the other period, and
the information processing device is configured to determine a display mode of the figure, based on the physiological information and reference information indicating a reference value for determining whether a value indicated by the physiological information is normal.

(3) The information processing device according to (1) or (2),
in which the information processing device is further configured to generate the plurality of pieces of change tendency information for each type of the physiological information, based on at least two or more types of the physiological information, and
in the display data, the plurality of pieces of change tendency information are displayed side by side or displayed so as to be overlapped with each other for each type of the physiological information.

(4) The information processing device according to any one of (1) to (3),
in which the plurality of pieces of change tendency information in the display data are displayed side by side, and
an arrangement order of the plurality of pieces of change tendency information indicates a temporal order of the change tendency information.

(5) The information processing device according to any one of (1) to (4),
in which the information processing device is further configured to:
   generate first change tendency information indicating a change tendency of the physiological information from a first period to a second period that is a period later in time than the first period, based on a first physiological information group and a second physiological information group, the first physiological information group including the physiological information during the first period, the second physiological information group including the physiological information during the second period,
   generate second change tendency information indicating a change tendency of the physiological information from the second period to a third period that is a period later in time than the second period, based on the second physiological information group and a third physiological information group, the third physiological information group including the physiological information during the third period, and
in the display data, the first change tendency information and the second change tendency information are displayed side by side or displayed so as to be overlapped with each other.

(6) The information processing device according to (5),
in which the information processing device is further configured to:
   generate the first change tendency information, based on a first comparison value calculated from a first value based on the first physiological information group and a second value based on the second physiological information group, and
   generate the second change tendency information, based on a second comparison value calculated from the second value and a third value based on the third physiological information group.

(7) The information processing device according to (6),
in which the first comparison value is a difference between the first value and the second value, and
the second comparison value is a difference between the second value and the third value.

(8) The information processing device according to (6) or (7),
in which the information processing device is configured to:
   change a display mode of the first change tendency information, according to the first comparison value, and
   change a display mode of the second change tendency information, according to the second comparison value.

(9) The information processing device according to any one of (6) to (8),
in which the first value is an average value of values obtained by performing moving average processing on the first physiological information group,
the second value is an average value of values obtained by performing moving average processing on the second physiological information group, and
the third value is an average value of values obtained by performing moving average processing on the third physiological information group.

(10) The information processing device according to any one of (5) to (7),
in which the display data includes a first axis that is a timeline and a second axis that indicates a value related to the physiological information, and
the information processing device is further configured to generate the display data in a form in which the first physiological information group, the second physiological information group, and the third physiological information group are displayed with the first axis overlapping and the second axis being common.

(11) The information processing device according to any one of (1) to (10),
in which the display data includes physiological waveform information.

(12) The information processing device according to any one of (1) to (11),
in which the change tendency information is displayed in an arrow shape in the display data.

(13) The information processing device according to any one of (1) to (12),
in which the display data includes name information indicating a name of the physiological information.

(14) The information processing device according to any one of (1) to (4) and (11) to (13),
in which the information processing device is further configured to generate:
   third change tendency information indicating a change tendency of the physiological information during a first period; and
   fourth change tendency information indicating a change tendency of the physiological information during a fourth period, and
the fourth period is included in the first period and is shorter than the first period.

(15) The information processing device according to any one of (1) to (13),
in which the information processing device is further configured to generate:
   first change tendency information indicating a change tendency of the physiological information from a first period to a second period that is a period later in time than the first period; and
   fifth change tendency information indicating a change tendency of the physiological information from a fourth period to a fifth period that is a period later in time than the fourth period, and
the fourth period and the fifth period are periods included in the first period and shorter than the first period.

(16) An information processing method executed by an information processing device, the information processing method including:
generating, based on physiological information on a subject, a plurality of pieces of change tendency information indicating a change tendency of the physiological information during one period or a change tendency of the physiological information from one period to another period; and
generating display data in which the plurality of pieces of change tendency information are displayed side by side or the plurality of pieces of change tendency information are displayed so as to be overlapped with each other.

(17) A non-transitory computer-readable medium storing a computer program including at least one instruction executed by one or more processors of an information processing device, the computer program causing, when the at least one instruction is executed by the one or more processors, the information processing device to
generate, based on physiological information on a subject, a plurality of pieces of change tendency information indicating a change tendency of the physiological information during one period or a change tendency of the physiological information from one period to another period, and
generate display data in which the plurality of pieces of change tendency information are displayed side by side or the plurality of pieces of change tendency information are displayed so as to be overlapped with each other.

What is claimed is:

1. An information processing device comprising:
at least one processor; and
one or more memories configured to store at least one instruction to be executed by the at least one processor,
wherein when the at least one instruction is executed by the processor, the information processing device is configured to:
generate, based on physiological information on a subject, a plurality of pieces of change tendency information indicating a change tendency of the physiological information during one period or a change tendency of the physiological information from one period to an other period; and
generate display data in which the plurality of pieces of change tendency information are displayed side by side or the plurality of pieces of change tendency information are displayed so as to be overlapped with each other.

2. The information processing device according to claim 1,
wherein in the display data, the change tendency information is displayed as a figure having an inclination,
the inclination indicates an amount of change of the physiological information during the one period or an amount of change of the physiological information from the one period to the other period, and
the information processing device is configured to determine a display mode of the figure, based on the physiological information and reference information indicating a reference value for determining whether a value indicated by the physiological information is normal.

3. The information processing device according to claim 1,
wherein the information processing device is further configured to generate the plurality of pieces of change tendency information for each type of the physiological information, based on at least two or more types of the physiological information, and
in the display data, the plurality of pieces of change tendency information are displayed side by side or displayed so as to be overlapped with each other for each type of the physiological information.

4. The information processing device according to claim 1,
wherein the plurality of pieces of change tendency information in the display data are displayed side by side, and
an arrangement order of the plurality of pieces of change tendency information indicates a temporal order of the change tendency information.

5. The information processing device according to claim 1,
wherein the information processing device is further configured to:
generate first change tendency information indicating a change tendency of the physiological information from a first period to a second period that is a period later in time than the first period, based on a first physiological information group and a second physiological information group, the first physiological information group including the physiological information during the first period, the second physiological information group including the physiological information during the second period,
generate second change tendency information indicating a change tendency of the physiological information from the second period to a third period that is a period later in time than the second period, based on the second physiological information group and a third physiological information group, the third physiological information group including the physiological information during the third period, and
in the display data, the first change tendency information and the second change tendency information are displayed side by side or displayed so as to be overlapped with each other.

6. The information processing device according to claim 5,
wherein the information processing device is further configured to:
generate the first change tendency information, based on a first comparison value calculated from a first value based on the first physiological information group and a second value based on the second physiological information group, and
generate the second change tendency information, based on a second comparison value calculated from the second value and a third value based on the third physiological information group.

7. The information processing device according to claim 6,
wherein the first comparison value is a difference between the first value and the second value, and
the second comparison value is a difference between the second value and the third value.

8. The information processing device according to claim 6,
wherein the information processing device is configured to:
change a display mode of the first change tendency information, according to the first comparison value, and
change a display mode of the second change tendency information, according to the second comparison value.

9. The information processing device according to claim 6,
wherein the first value is an average value of values obtained by performing moving average processing on the first physiological information group,
the second value is an average value of values obtained by performing moving average processing on the second physiological information group, and
the third value is an average value of values obtained by performing moving average processing on the third physiological information group.

10. The information processing device according to claim 5,
wherein the display data includes a first axis that is a timeline and a second axis that indicates a value related to the physiological information, and
the information processing device is further configured to generate the display data in a form in which the first physiological information group, the second physiological information group, and the third physiological information group are displayed with the first axis overlapping and the second axis being common.

11. The information processing device according to claim 1,
wherein the display data includes physiological waveform information.

12. The information processing device according to claim 1,
wherein the change tendency information is displayed in an arrow shape in the display data.

13. The information processing device according to claim 1,
wherein the display data includes name information indicating a name of the physiological information.

14. The information processing device according to claim 1,
wherein the information processing device is further configured to generate:
third change tendency information indicating a change tendency of the physiological information during a first period; and
fourth change tendency information indicating a change tendency of the physiological information during a fourth period, and
the fourth period is included in the first period and is shorter than the first period.

15. The information processing device according to claim 1,
wherein the information processing device is further configured to generate:
first change tendency information indicating a change tendency of the physiological information from a first period to a second period that is a period later in time than the first period; and
fifth change tendency information indicating a change tendency of the physiological information from a fourth period to a fifth period that is a period later in time than the fourth period, and
the fourth period and the fifth period are periods included in the first period and shorter than the first period.

16. An information processing method executed by an information processing device, the information processing method comprising:
generating, based on physiological information on a subject, a plurality of pieces of change tendency information indicating a change tendency of the physiological information during one period or a change tendency of the physiological information from one period to another period; and
generating display data in which the plurality of pieces of change tendency information are displayed side by side or the plurality of pieces of change tendency information are displayed so as to be overlapped with each other.

17. A non-transitory computer-readable medium storing a computer program including at least one instruction executed by one or more processors of an information processing device, the computer program causing, when the at least one instruction is executed by the one or more processors, the information processing device to:
generate, based on physiological information on a subject, a plurality of pieces of change tendency information indicating a change tendency of the physiological information during one period or a change tendency of the physiological information from one period to another period, and
generate display data in which the plurality of pieces of change tendency information are displayed side by side or the plurality of pieces of change tendency information are displayed so as to be overlapped with each other.
